(19) United States
(12) Patent Application Publication
(10) Pub. No.: US 2026/0168011 A1
(43) Pub. Date: Jun. 18, 2026

(54) Methods of Preparing and Analyzing Nucleic Acid Libraries

(71) Applicant: Takara Bio USA, Inc., San Jose, CA (US)

(72) Inventors: Emmanuel Kamberov, Ann Arbor, MI (US); Yoshitaka Kimura, Ann Arbor, MI (US); Julie Catherine Laliberté, Ann Arbor, MI (US); Patrick Kevin Martin, San Jose, CA (US); Jacob Meyers, Ann Arbor, MI (US)

(21) Appl. No.: 19/362,007

(22) Filed: Oct. 17, 2025

Related U.S. Application Data
(63) Continuation of application No. 17/276,771.
(60) Provisional application No. 62/806,698, filed on Feb. 15, 2019.

(51) Int. Cl. C12Q 1/6827; C12Q 1/6853

(52) U.S. Cl. CPC C12Q 1/6827; C12Q 1/6853

(57) ABSTRACT

Detecting different mutations in the same sample is essential, especially where the sample is limited in quantity and where high-throughput methods are desired for rapid detection of mutations. Methods routinely used in the art require separate assays for detecting different mutations or mutation types (e.g. single nucleotide polymorphisms (SNPs) or copy number variations (CNVs)) in a sample. The present disclosure provides methods for detecting different mutations, such as SNPs and CNVs in the same sample. The methods described herein can be useful in pre-implantation genetic testing, carrier screening, or genotyping.

### CROSS-REFERENCE TO RELATED APPLICATION

This application is a continuation of U.S. patent application Ser. No. 17/276,771 filed Mar. 16, 2021, which application is a U.S. National Stage Application of PCT Application No. PCT/US2020/018360 filed Feb. 14, 2020, which application, pursuant to 35 U.S.C. § 119 (e), claims priority to the filing date of U.S. Provisional Patent Application Ser. No. 62/806,698 filed Feb. 15, 2019; the disclosures of which applications are herein incorporated by reference.

### INTRODUCTION

Detecting different mutations in a same sample is essential, especially where the sample is limited in quantity and where high-throughput methods are desired for rapid detection of mutations. Methods routinely used in the art require separate assays for detecting different mutations or mutation types (e.g., single nucleotide polymorphisms (SNPs) or copy number variations (CNVs)) in a sample. Using separate assays may pose a risk of missing clinically significant mutations in samples with limited quantities.

### SUMMARY

The present disclosure provides methods for detecting different mutations, such as SNPs and CNVs in the same sample. The methods described herein can be useful in pre-implantation genetic testing, carrier screening, or genotyping.

In an aspect, the present disclosure provides a method of detecting single nucleotide polymorphism (SNP) and copy number variation (CNV) in a sample. The method comprises a) obtaining a sample comprising nucleic acid molecules; b) subjecting the nucleic acid molecules to a population of primers for whole genome amplification or whole transcriptome amplification and to at least one target-specific primer for targeted amplification to generate a mixture of amplicons produced by the whole genome amplification or whole transcriptome amplification and the targeted amplification; c) sequencing the mixture of amplicons using a sequencing assay on a sequencer to generate sequencing reads; and d) assessing the sequencing reads to determine the SNP and CNV in the sample.

In some embodiments, the nucleic acid molecules are amplified by using a polymerase chain reaction. In some embodiments, the plurality of nucleic acid molecules is at least 50 base pairs. In some embodiments, the nucleic acid molecules comprise genomic DNA, or RNA.

In some embodiments, the mixture of amplicons produced in step (b) is subjected to an additional targeted amplification using at least one nested primer pair to further amplify amplicons generated by the targeted amplification.

In some embodiments, the method further comprises using the sequencing reads to genotype single nucleotide variation (SNV), genotype micro-satellite, detect insertion and/or deletion, determine zygosity, determine sex, detect gene fusions, detect translocation(s), detect mutation(s), or detect chromosomal abnormalities.

In some embodiments, the population of primers are non-self-complementary and non-complementary to other primers in the population, and comprise in a 5′ to 3′ orientation a constant region and a variable region, wherein the constant region sequence has a known sequence that is constant among a plurality of primers of the population and the variable region sequence is degenerate among the plurality of primers of the population, and further wherein the sequence of the constant and variable regions consists will not cross-hybridize or self-hybridize under conditions to carry out steps (a)-(c).

In some embodiments, the primers as in (b) comprise at least 10 nucleotides. In some embodiments, the at least one target-specific primer is specific to one or more target sequences. In some embodiments, the at least one target-specific primer does not comprise an adapter sequence. In some embodiments, the at least one target-specific primer comprises at least a portion of an adapter sequence. In some embodiments, the primers as in (b) comprises at least one modified nucleotide. In some embodiments, melting temperature of the primers as in (b) is at least 30 degrees Celsius. In some embodiments, the at least one target-specific primer comprises a single target-specific primer pair. In some embodiments, the one or more target sequences comprise a redundant genomic region. In some embodiments, the redundant genomic region comprises a repetitive element. In some embodiments, the repetitive element comprises an SVA element.

In some embodiments, the sample is selected from the group consisting of blood, serum, plasma, cerebrospinal fluid, cheek scrapings, nipple aspirate, biopsy, cervical sample, semen, bodily fluid, microorganisms, mitochondria, chloroplasts, a cell lysate, urine, feces, hair follicle, saliva, sweat, immunoprecipitated or physically isolated chromatin, circulating tumor cells, tumor biopsy samples, exosomes, embryo, cell culture medium, spent medium for culturing cells, tissues, organoids, or embryos, biopsied embryo, trophoblast, amniotic fluid, maternal blood, fetal cell, fetal DNA, cell-free DNA, uterine lavage fluid, endometrial fluid, cumulus cells, granulosa cells, formalin-fixed tissue, paraffin-embedded tissue or blastocoel cavity.

In an aspect, the present disclosure provides a kit The kit comprises a) a population of primers for whole genome amplification or whole transcriptome amplification; b) at least one target-specific primer for targeted amplification; and d) a set of instructions for using the kit to detect copy number variation (CNV), genotype single nucleotide polymorphism (SNP), detect single nucleotide variation (SNV), genotype micro-satellite, detect insertion and/or deletion, determine zygosity, determine sex, detect gene fusions, detect translocations, detect mutation(s), or detect chromosomal abnormalities.

### BRIEF DESCRIPTION OF THE FIGURES

FIG. 1 provides a schematic representation of a method for conducting whole genome amplification (WGA) using WGA primers for detecting copy number variations (CNVs) and targeted amplification using target-specific primers for detecting single nucleotide polymorphisms (SNPs) using a same nucleic acid sample.

FIG. 2 provides an example of a protocol for preparing nucleic acid molecules to detect a copy number variation (CNV) and a single nucleotide polymorphism (SNP) by respectively carrying out whole genome amplification (WGA) and targeted amplification using a same sample of nucleic acid molecules.

FIG. 3 provides a schematic representation of steps for generating nucleic acid library molecules for the detection of SNPs and CNVs using the same sample of nucleic acid molecules. The steps may include a pre-amplification step with WGA and targeted amplification, an optional clean-up step, one or more library preparation steps such as a targeted amplification step using nested PCR, and an indexing PCR step to generate nucleic acid library molecules for sequencing.

FIG. 4 provides a schematic of an embodiment of a redundant genomic element.

FIG. 5 provides a schematic of a SINE/VNTR/Alu (SVA) element (FIG. 5A) and a schematic representation of target-specific primers complementary to regions of an SVA element (FIG. 5B).

FIG. 6 provides a schematic representation of a method for detecting SNPs and CNVs using the same sample of nucleic acid molecules by performing whole genome amplification (WGA) using WGA primers and targeted amplification using target-specific primers complementary to redundant genomic elements.

FIG. 7 provides a schematic of using multiple target-specific primers spanning the target sequence.

FIG. 8A and FIG. 8B provide data from an experiment performed using three different pre-amplification conditions, namely, without target-specific primers, with 30 target-specific primers and with 90 target-specific primers. FIG. 8A shows the coverage with three pre-amplification conditions. FIG. 8B shows variation in the coverages, as indicated by the coefficient of variation, among three pre-amplification conditions.

FIG. 9A, FIG. 9B, FIG. 9C, and FIG. 9D provide data from an experiment where pre-amplification was carried out with or without targeted amplification. In either case, i.e. with or without targeted amplification in the pre-amplification step, targeted amplification was carried out after the pre-amplification step. FIG. 9A shows the percentage of reads spanning the whole genome and the target sequence i.e., the CFTR gene, using assays with or without targeted amplification in the pre-amplification step. FIG. 9B shows the average coverage for the whole genome and the CFTR gene with or without targeted amplification in the pre-amplification step. FIG. 9C shows the coverage of sequencing reads across the fifteen different targets or variants in the CFTR gene from an assay where the pre-amplification reaction included targeted amplification while FIG. 9D shows the coverage from an assay where the pre-amplification reaction did not include targeted amplification.

FIG. 10A and FIG. 10B provide the coverage data of sequencing reads from an experiment performed using 5 cells (FIG. 10A) or a single cell (FIG. 10B).

FIG. 11A and FIG. 11B show the distribution of sequencing reads from an experiment performed using 5 cells (FIG. 11A) or a single cell (FIG. 11B).

FIG. 12A, FIG. 12B, and FIG. 12C provide data from an experiment to assess correlation among replicates using five cell replicates (FIG. 12A) or single cell replicates (FIG. 12B). FIG. 12C shows the genomic view of the log 2 ratio of reads in 1 Mb bins in two replicates.

FIG. 13 provides data from an experiment to show the coverage across 15 different targets on the CFTR gene using targeted amplification only without WGA for carrier screening, for example.

FIG. 14A, FIG. 14B, FIG. 14C, and FIG. 14D provide a schematic of comparison between the traditional method (FIG. 14A) and the present method (FIG. 14B) as well as data using the present method (FIG. 14C and FIG. 14D) for detecting single nucleotide polymorphisms (SNPs) in the CFTR gene and chromosomal aneuploidy in trophectoderm biopsies (n=4). FIG. 14C provides data related to the detection of SNPs in the CFTR gene and FIG. 14D provides data related to the detection of aneuploidies using the present method.

FIG. 15 provides a visual representation of SNPs found within SVA elements across the human genome (assembly hg38). Top bar represents individual chromosomes 1-22, X & Y. Bottom graph depicts individual SNPs as dots across the genome. Y-axis represents the minor allele frequency of each SNP. Black dots represent SNPs with a minor allele frequency greater than or equal to 0.05. Grey dots represent SNPs with a minor allele frequency below 0.05.

FIG. 16 provides embodiments of target-specific primer pairs and a number of predicted PCR products or amplicons for each primer pair. The sequences are set forth as follows: Alu-like Primer Sequences from top to bottom (SEQ ID NOs: 1-10); SINE-R Primer Sequences from top to bottom (SEQ ID NOs: 11-20).

### DETAILED DESCRIPTION

Methods of preparing and analyzing nucleic acid molecules by amplifying whole genome or transcriptome (WGA or WTA) in combination with targeted amplification to amplify whole genome and target sequences from the same sample of nucleic acid molecules are provided. The methods can be useful in the detection of various mutations, such as copy number variations (CNVs), insertion and/or deletion (indel) and single nucleotide polymorphisms (SNPs) in the same sample. The methods find use in clinical testing, (e.g., carrier screening, embryo screening, spent media testing), forensic analysis, etc.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

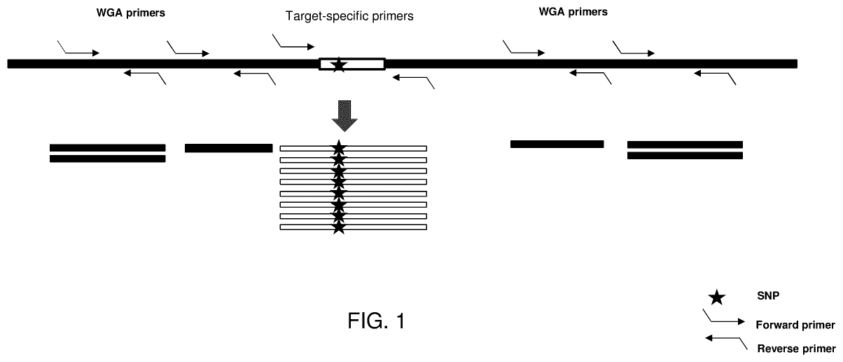

As summarized above, the methods described in this disclosure relate to preparing and analyzing nucleic acid molecules for detecting various mutations (e.g., copy number variation and single nucleotide polymorphisms) in a same sample, blood, cells, spent media, or extracted nucleic acid, for example. Broadly, the methods include amplifying nucleic acid molecules using primers for whole genome amplification (WGA) or whole transcriptome amplification (WTA) in combination with and/or followed by targeted amplification of target sequence(s) using target-specific primers. For example, as shown in FIG. 1, whole genome amplification (WGA) using WGA primers for detecting copy number variations (CNVs) in combination with targeted amplification using target-specific primers, encompassing a SNP, for detecting SNPs can be carried out using the same nucleic acid sample.

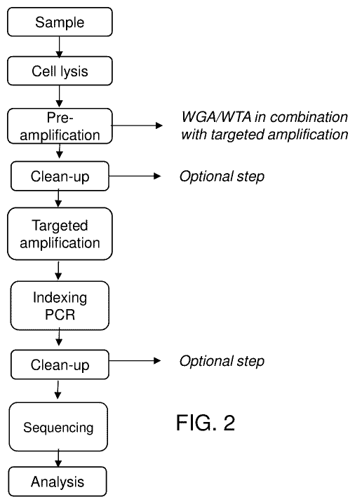

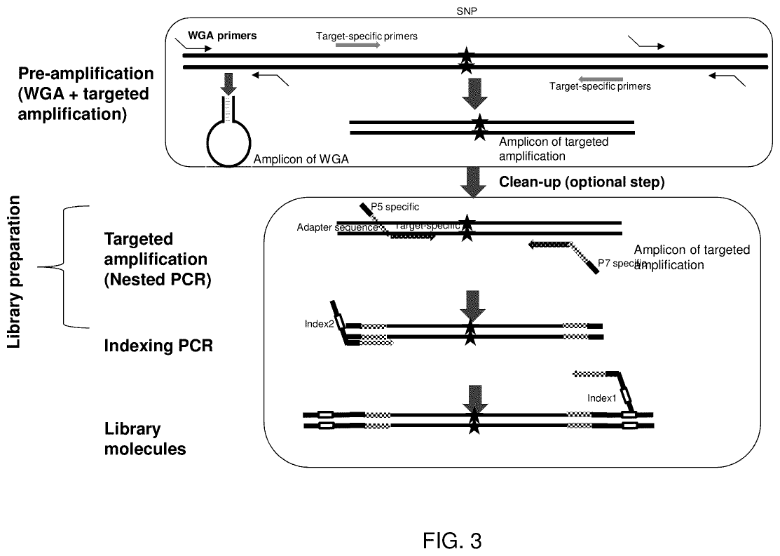

The methods disclosed herein can include various steps. An example of one such protocol is provided in FIG. 2 and FIG. 3. The protocol can include steps, such as obtaining a sample comprising nucleic acid molecules, lysing the sample to extract nucleic acid molecules from the sample, subjecting the nucleic acid molecules to a pre-amplification step to amplify whole genome using WGA/WTA primers in combination with targeted amplification to amplify target sequence(s) using target-specific primers, optionally performing a clean-up step followed by subjecting amplicons to a library preparation procedure to prepare library molecules for sequencing. The library preparation step can include one or more steps to attach sequences necessary for a sequencing assay. The library preparation step may include more than one step, for example, where the pre-amplification step does not include targeted amplification, or the pre-amplification step includes targeted amplification, but an additional targeted amplification may be applied following the pre-amplification step. In cases where an additional targeted amplification is carried out following the pre-amplification step, nested PCR may be performed to further amplify target sequence(s) and to attach adapter sequences (e.g., P5 or P7). The nested PCR may be carried out using primers that are nested within the target-specific primers used in the pre-amplification step. In some cases, the library can be prepared in a single step to attach adapter sequences and indices in a single reaction. For example, the pre-amplification step may include targeted amplification and an additional targeted amplification following the pre-amplification step may be optional. In this case, the library can be prepared in a single step, for example, during indexing PCR. An indexing PCR can be carried out either following the pre-amplification step and/or targeted amplification, to attach indices (e.g., Index 1 or Index 2) to the amplicons. Various steps of the methods are described in FIGS. 1-3 and in greater detail below.

### Sample

The methods in this disclosure can be used with a variety of samples comprising nucleic acid molecules, such as DNA or RNA. In some cases, a sample can be blood, serum, plasma, cerebrospinal fluid, cheek scrapings, cervical fluid/cells, nipple aspirate, biopsy, semen, urine, feces, hair follicle, saliva, sweat, immunoprecipitated or physically isolated chromatin, circulating tumor cells, tumor biopsy, exosomes, an embryo, cell culture medium, spent medium for culturing cells, tissues, organoids, or embryos, a biopsied embryo (such as one or more cells from the inner cell mass (ICM) of a blastocyst or one or more cells from the trophectoderm (TE)—i.e., trophectoderm cells), amniotic fluid, formalin-fixed tissue, maternal blood, fetal cell(s), cell-free DNA, uterine lavage fluid, endometrial fluid, cumulus cells, granulosa cells, cancer cell(s), paraffin-embedded tissue or blastocoel cavity. In some cases, a sample can be an oocyte or a polar body thereof, microorganisms, plant cells, animal cells, mitochondria, chloroplasts, a forensic sample, a cell lysate, bodily fluid, a cervical sample. Other types of samples comprising nucleic acid molecules can also be used.

### Cell Lysis and Extraction of Nucleic Acid Molecules

A sample comprising nucleic acid molecules can be lysed to release nucleic acid molecules. In some cases, the sample can be lysed using any methods known in the art, such as reagent-based methods and physical methods. For example, the reagent-based methods can include using enzymes (e.g., lysozyme), and/or organic solvents (e.g., alcohols, chloroform, ethers, EDTA, triton, alkaline lysis). Examples of the physical methods can include sonication, homogenizer, freeze-thaw cycles, grinding, etc. In some cases, cell lysis may not be required, and the sample can be directly used for preparing nucleic acid molecules using the methods disclosed herein. For example, the sample can be cell-free DNA that can be used with the methods in this disclosure.

In some embodiments, the amount/quantity of nucleic acid molecules that can be used with the methods described herein can be at least 0.5 picogram (pg), at least 1 pg, at least 2 pg, at least 5 pg, at least 10 pg, at least 20 pg, at least 30 pg, at least 40 pg, at least 50 pg, at least 100 pg, at least 200 pg, at least 500 pg, at least 1 nanogram (ng), or more than 1 ng. Other amounts can be used with the methods in this disclosure.

In some embodiments, the quality of nucleic acid molecules that can be used with the methods in this disclosure can be high-quality nucleic acid molecules without significant amounts of inhibitors, such as extracted DNA using the methods disclosed in the art. In some cases, the sample of nucleic acid molecules can include inhibitors, such as formalin-fixed samples.

### Pre-Amplification

Nucleic acid molecules can be subjected to a pre-amplification step. The pre-amplification step can include subjecting nucleic acid molecules to the primers for whole genome amplification (WGA) or whole transcriptome amplification (WTA). In some embodiments, the pre-amplification step may include target-specific primers for targeted amplification to generate a mixture of amplicons from WGA/WTA and targeted amplification. In some cases, the pre-amplification step may not include target-specific primers and as such, the pre-amplification step may generate amplicons from WGA only. In this case, the pre-amplification step may be followed by targeted amplification to amplify target sequence(s) using target-specific primers. In embodiments where the pre-amplification reaction may include WGA/WTA primers in combination with target-specific primers to generate a mixture of amplicons, the mixture of amplicons may further be subjected to targeted amplification using primers nested within the amplicons produced by targeted amplification in the pre-amplification step. In some specific embodiments, the pre-amplification step may not be carried out. In this case, nucleic acid molecules are subjected to targeted amplification to amplify target sequence(s) using target-specific primers.

WGA or WTA can substantially amplify all fragments of the nucleic acid molecules in a sample. WGA or WTA can substantially amplify entire genome or entire transcriptome without loss of representation of specific sites. Substantially all or substantially entire can refer to about 30%, about 40%, about 50%, about 60%, about 70%, about 80%, about 85%, about 90%, about 95%, or more of all sequences in a genome or transcriptome. WGA or WTA, in some cases, can include non-equivalent amplification of particular sequences over others, although the relative difference in such amplification is not considerable in some cases. WGA/WTA can target one or more sequences in the genome or transcriptome. In most instances, WGA/WTA can target at least about 100, at least about 1000, at least about 10,000, at least about 100,000, at least about 1,000,000, at least about 10,000,000, at least about 100,000,000, at least about 1,000,000,000, or more sites in the genome or transcriptome. WGA and/or WTA may be performed with any suitable primers. Suitable WGA/WTA primers include, but are not limited to, primers provided in a PicoPLEX® WGA kit, SMARTer® PicoPLEX® Single Cell WGA kit, SMARTer® PicoPLEX® DNA-seq kit, SMARTer® PicoPLEX® Gold Single Cell DNA-Seq kit, lon ReproSeq™ PGS kit, MALBAC® Single Cell WGA kit, GenomePlex® WGA kits, REPLI-g® WGA and WTA kits, Ampli1™ WGA and WTA kits, Transplex® WTA kits, TruePrime® WGA kits, Quantitect® Whole Transcriptome kit, Doplify® WGA kit, GenoMatrix™ WGA kit, PG-Seq™ kit, Sureplex™ DNA Amplification System kit, Illustra GenomiPhi™ DNA Amplification kit. Suitable WGA/WTA primers may be described in, for example, U.S. Pat. Nos. 7,718,403; 8,206,913; 9,249,459; 9,617,598; 5,731,171; 6,365,375; 10,017,761; 8,034,568; 6,617,137; 6,977,148, 10,190,163; 9,840,732; 9,777,316; 8,512,956; 8,349,563, the contents of each of which are incorporated by reference herein, and U.S. Patent Publication Nos. 2016/0355879; 2018/0030522; 2019/0271033; 2013/0085083; 2007/0054311; 2007/0178457; 2011/0033862; 2016/0312276; 2009/0099040; 2010/0184152; 2015/0072899; 2011/0189679; 2019/0300933; 2016/0289740, the contents of each of which are incorporated by reference herein.

Similarly, target-specific primers can amplify one or more sequences in the genome or transcriptome during targeted amplification. In some cases, target-specific primers can amplify one sequence, 2 sequences, 3 sequences, 10 sequences, 100 sequences, 1000 sequences, 10,000 sequences, 100,000 sequences, 1,000,000 sequences, 10,000,000 sequences, or more. In some cases, targeted amplification can amplify the same sequence using one or more target-specific primers. In other cases, targeted amplification can amplify different sequences in the genome or transcriptome. In some cases, a “target-specific primer” refers to a primer that hybridizes selectively and predictably to a target sequence under suitable conditions for hybridization. In some cases, a “target sequence” or “target sequence of interest” and its derivatives, refers generally to any single or double-stranded nucleic acid sequence that can be amplified according to the disclosure, including any nucleic acid sequence suspected or expected to be present in a sample. In some embodiments, the target sequence is present in double-stranded form and includes at least a portion of the particular nucleotide sequence to be amplified or synthesized, or its complement, prior to the addition of target-specific primers. Target sequences can include the nucleic acids to which the target-specific primers can hybridize prior to extension by a polymerase. In some cases, the target-specific primers amplify a target sequence including one or more mutational hotspots, genomic markers, SNPs of interest, redundant genomic elements (e.g., SVA elements), coding regions, exons, genes, introns, non-coding regions, promoter regions, pseudogene, intron-exon junction, and intergenic regions. In some cases, the target-specific primers can amplify target sequences including one or more genomic regions of interest such as, e.g., genes of interest (e.g., the CFTR gene) or one or more regions of a gene of interest. In some cases, target-specific primers can amplify target sequences including one or more SNPs of interest. In some cases, target-specific primers can amplify target sequences including genes or genomic regions implicated in genetic disorders such as any of the genetic disorders disclosed herein.

In certain embodiments, the one or more target sequences of the target-specific primers include a redundant genomic region or redundant genomic element, i.e., a genomic region present throughout the genome, e.g., of a human. The redundant genomic region may be present on all chromosomes, e.g., in an even manner. In some cases, the redundant genomic region is present at multiple locations in the genome such as, e.g., 1000 or more locations in the genome, 2000 or more locations in the genome, 3000 or more locations in the genome, 4000 or more locations in the genome, 5000 or more locations in the genome, 6000 or more locations in the genome, 7000 or more locations in the genome, 8000 or more locations in the genome, 9000 or more locations in the genome, 10,000 or more locations in the genome, 100,000 or more locations in the genome, 1,000,000 or more locations in the genome, 10,000,000 or more locations in the genome, or 100,000,000 or more locations in the genome. In some cases, the redundant genomic region is present in multiple locations in the genome ranging from 1000 to 10,000,000 locations in the genome, from 1000 to 1,000,000 locations in the genome, from 10,000 to 500,000 locations in the genome, or from 50,000 to 200,000 locations in the genome.

The genomic regions present in multiple locations in a genome may be diverse in sequence, e.g., such that the genomic regions uniquely map across the genome. In some cases, the redundant genomic region is polymorphic (e.g., includes SNPs). As used herein in its conventional sense, “polymorphic” refers to the condition in which two or more variants of a specific genomic sequence can be found in a population. In some cases, the redundant genomic region includes one or more polymorphic regions. The polymorphic regions may include insertions, deletions, structural variant junctions, variable length tandem repeats, single nucleotide mutations, single nucleotide variations, copy number variations, or a combination thereof. In some cases, the polymorphic regions have a minor allele frequency ranging from 0.01 or greater, from 0.02 or greater, from 0.03 or greater, from 0.04 or greater, from 0.05 or greater, from 0.06 or greater, from 0.07 or greater, from 0.08 or greater, from 0.09 or greater, from 0.1 or greater, from 0.2 or greater, from 0.3 or greater, or from 0.4 or greater. In some cases, the one or more polymorphic regions provide one or more SNPs per region such as, e.g., 1-5 SNPs per region, 10-20 SNPs per region, 10-40 SNPs per region, 15-35 SNPs per region, 20-60 SNPs per region, or 20-50 SNPs per region. In some cases, the redundant genomic region includes one or more conserved regions. As used herein in its conventional sense, a “conserved region” refers to a region in heterologous polynucleotide or polypeptide sequences or polynucleotide or polypeptide sequences that are present in different species or duplicated within a genome where there is a relatively high degree of sequence identity between the distinct sequences. The sequence identity between the conserved regions may be at least 75%, at least 80%, at least 85%, at least 90%, at least 95%, at least 98%, or at least 99%. In some cases, the redundant genomic region includes a polymorphic region flanked on both ends by conserved regions. In some cases, the redundant genomic regions include non-coding regions of the genome. Genomic regions of interest may include, for example, one or more introns, one or more regulatory elements, one or more pseudogenes, one or more repeat sequences or repetitive elements, one or more viral elements (e.g., endogenous retrovirus sequences), one or more telomeres, one or more transposable elements, one or more retrotransposons, one or more short tandem repeats, a portion thereof or a combination thereof.

The redundant genomic region may have any length suitable for amplification by the subject methods. In some cases, the redundant genomic region has a length ranging from 1000 to 4000 base pairs (bp), from 1000 to 3000 bp, from 1000 to 2000 bp, or from 500 to 1500 bp. In some cases, the genomic region has a length ranging from 1 to 500 base pairs (bp), from 10 to 500 bp, or from 100 to 500 bp.

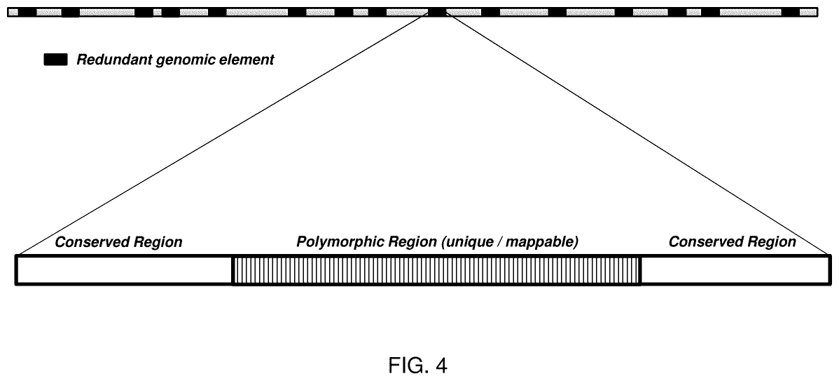

FIG. 4 provides a schematic of an embodiment of a redundant genomic element that is present across the genome in multiple locations (top) and a schematic of the embodiment of the redundant genomic element having a polymorphic region flanked by conserved regions on both ends (bottom). The redundant genomic element may be found throughout the genome and is present on all chromosomes in a relatively even manner. The genome may include 1500-3000 copies or more, 3000-30000 copies or more, 30000-300000 copies or more of the redundant genomic element, which amounts to approximately one region of SNPs for every 1-2 Mb of the genome.

In some cases, the redundant genomic region includes a repetitive element or repeat sequence. Repetitive elements may include one or more tandem repeats, one or more interspersed repeats, or a combination thereof.

Tandem repeats may include one or more satellite DNA, one or more minisatellites (long tandem repeats; repeat unit of 10-100 bp), one or more microsatellites (short tandem repeats; repeat units of less than 10 bp) or a combination thereof. In some cases, the redundant genomic region includes a VNTR (variable number tandem repeat). In some cases, the redundant genomic region includes macrosatellites (repeat unit is longer than 100 bp).

Interspersed repeats may be dispersed across the genome within gene sequences or intergenic. Interspersed repeats may include one or more transposons. Transposons may be mobile genetic elements. Mobile genetic elements may change their position within the genome. Transposons may be classified as class I transposable elements (class I TEs) or class II transposable elements (class II TEs). Class I TEs (e.g., retrotransposons) may copy themselves in two stages, first from DNA to RNA by transcription, then from RNA back to DNA by reverse transcription. The DNA copy may then be inserted into the genome in a new position. Class I TEs may comprise one or more long terminal repeats (LTRs), one or more long interspersed nuclear elements (LINEs), one or more short interspersed nuclear elements (SINEs), or a combination thereof. Examples of LTRs include, but are not limited to, human endogeneous retroviruses (HERVs), medium reiterated repeats 4 (MER4), and retrotransposon. Examples of LINES include, but are not limited to, LINE1 and LINE2. SINEs may comprise one or more Alu sequences, one or more mammalian-wide interspersed repeat (MIR), or a combination thereof. Class II TEs (e.g., DNA transposons) often do not involve an RNA intermediate. The DNA transposon is often cut from one site and inserted into another site in the genome. Alternatively, the DNA transposon is replicated and inserted into the genome in a new position. Examples of DNA transposons include, but are not limited to, MER1, MER2, and mariners.

Interspersed repeats may include one or more retrotransposable elements. Retrotransposable elements (REs), include long interspersed nuclear elements (LINEs), short interspersed nuclear elements (SINEs) and SVA elements. SINEs are a class of REs that are typically less than 500 nucleotides long; while LINEs are typically greater than 500 nucleotides long (A. F. A. Smit, The origin of interspersed repeats in the human genome, Current Opinion in Genetics Development, 6(6): 743-748(1996); Batzer, M. A., et al., Alu repeats and human genomic diversity, Nature Reviews Genetics, 3(5): 370-379 (2002); Batzer, M. A., et al., African origin of human-specific polymorphic Alu insertions, Proceedings of the National Academy of Sciences, 91(25): 12288(1994); Feng, Q., et al., Human L1 retrotransposon encodes a conserved endonuclease required for retrotransposition, Cell, 87(5): 905-916(1996); Houck, C. M., et al., A ubiquitous family of repeated DNA sequences in the human genome, Journal of Molecular Biology, 132(3): 289-306 (1979); Kazazian, H. H., et al., The impact of L1 retrotransposons on the human genome, Nature Genetics, 19(1): 19-24 (1998); Ostertag, E. M., et al., Biology of mammalian L1 retrotransposons, Annual Review of Genetics, 35(1): 501-538 (2001)). LINE full-length elements are approximately 6 kb in length, contain an internal promoter for polymerase II and two open reading frames (ORFs) and end in a polyA-tail. SINEs include Alu elements, primate specific SINEs that have reached a copy number in excess of one million in the human genome. SINEs were originally defined by their interspersed nature and length (75-500 bp), but have been further characterized by their RNA polymerase III transcription.

The third type of RE is the composite retrotransposon known as an SVA (SINE/VNTR/Alu) element (Wang, H., et al., SVA Elements: A Hominid-specific Retroposon Family, J. Mol. Biol. 354:994-1007 (2005). SVAs are evolutionarily young and presumably mobilized by the LINE-1 reverse transcriptase in trans. SVAs are currently active and may impact the host through a variety of mechanisms including insertional mutagenesis, exon shuffling, alternative splicing, and the generation of differentially methylated regions (DMR). Each domain of SVA is derived from either a retrotransposon or a repeat sequence. A canonical SVA is on average ˜2 kilobases (kb) (e.g., ˜1,650 bp), but SVA insertions may range in size from 700-4000 basepairs (bp) (Hancks, D. C., and Kazazian, H. H., SVA Retrotransposons: Evolution and Genetic Instability, Semin. Cancer Biol. 20:234-45 (2010)). SVAs are composite elements named after their main components, SINE, a variable number of tandem repeats (VNTR), and Alu. SVA elements contain the hallmarks of retrotransposons, in that they are flanked by target site duplications (TSDs), terminate in a poly(A) tail and are occasionally truncated and inverted during their integration into the genome. Canonical SVAs typically contain five distinct regions; a (CCCTCT), (SEQ ID NO: 25) hexamer repeat at the 5′ end, an Alu-like domain, a variable number tandem repeat (VNTR), a SINE-derived region (e.g., SINE-R where R indicates retroviral origin), and a poly(A) tail. As a consequence of the repetitive domains, e.g., VNTR region, full-length SVA elements can vary greatly in size. SVAs may be categorized into six subfamilies named SVA_A, SVA_B, SVA_C, SVA_D, SVA_E, SVA_F. The homology of the families ranges from 90-95% using a family-wise consensus sequence. In a seventh subfamily SVA-F1, the (CCCTCT) n (SEQ ID NO: 25) hexamer is replaced by a 5′ transduction of the first exon of the MAST2 gene (Quinn, J., et al., The Role of SINE-VNTR-Alu (SVA) Retrotransposons in Shaping the Human Genome, Int. J. Mol. Sci. 20:5977 (2019)).

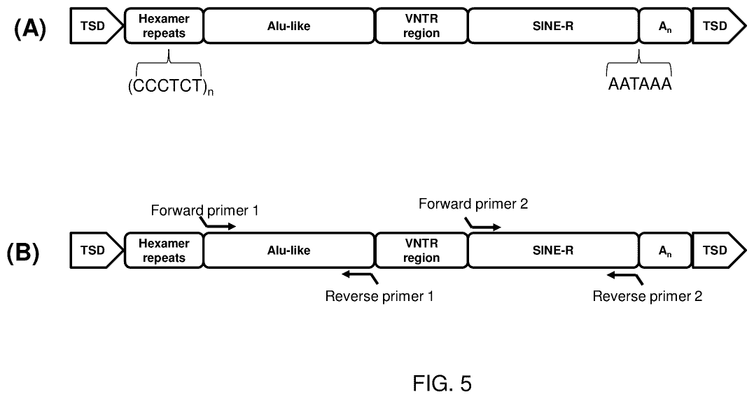

In some cases, SVA elements are polymorphic (e.g., include SNPs). The polymorphic regions of SVA elements may include one or more of any of the domains and regions of SVA elements described herein. In some cases, the Alu-like domain of SVA elements is polymorphic. In some cases, the SINE-R region of SVA elements is polymorphic. In some cases, the conserved regions of SVA elements include one or more of the target site duplication domains, the hexamer repeat, VNTR, and poly-A tail. An embodiment of an SVA element is provided in FIG. 5A (adapted from Wang, H., et al., SVA Elements: A Hominid-specific Retroposon Family, J. Mol. Biol. 354:994-1007 (2005)). The SVA element includes two flanking target site duplication domains, a hexamer repeat (CCCTCT)ₙ (SEQ ID NO: 25), an Alu-like domain including two partial Alu elements connected by SVA-U (335 nt), a VNTR region (varies from 48-2,306 bp; mean length: 819 bp), a SINE-R region made of segments from human endogenous retrovirus (env, U3, R) (490 nt), and a poly-A tail.

In some cases, the redundant genomic region includes a pseudogene. “Pseudogene” and “pseudogenes,” as used herein, refer to sequences that have a high sequence similarity or sequence identity to identified genes but are generally untranscribed and untranslated due to non-functional promoters, missing start codons or other defects. Most pseudogenes are intronless and represent mainly the coding sequence of the parent gene. For some cases, it has been shown that in different organisms or tissues functional activation may occur.

In some cases, the targeted amplification as described above includes amplifying a target sequence using one or more target-specific primer pairs. In some cases, the one or more target-specific primer pairs include fifty or less primer pairs, fifteen or less primer pairs, ten or less primer pairs, nine or less primer pairs, eight or less primer pairs, seven or less primer pairs, six or less primer pairs, five or less primer pairs, four or less primer pairs, three or less primer pairs, two or less primer pairs, or a single primer pair. In certain embodiments, the subject methods include amplifying nucleic acid molecules using primers for WGA/WTA in combination with and/or followed by at least one target-specific primer, where the at least one target-specific primer includes a single target-specific primer pair.

In some cases, the target-specific primers for targeted amplification in the subject methods include a single primer pair for amplifying a redundant genomic region as described above. In some cases, the primers of the single primer pair are specific to or complementary to a redundant genomic region or one or more portions of a redundant genomic region, e.g., a polymorphic region of the redundant genomic region. In some cases, the primers of the single primer pair are specific to one or more regions or domains of a repetitive element, e.g., an SVA element. In FIG. 5B, the primers of a primer pair complementary to portions of the Alu-like domain or to portions of the SINE-R domain are provided. In some cases, one or more primers of the single primer pair are complementary to the Alu-like domain of the SVA element or a portion of the Alu-like domain. In some cases, one or more primers of the single primer pair are complementary to the SINE-R region of the SVA element or a portion of the SINE-R region. In some cases, the subject methods including targeted amplification using a single primer pair specific to a redundant genomic element, e.g., an SVA element, in addition to WGA/WTA quasi-random primers find use in SNP-based CNV calling, detecting uniparental disomy, detecting chromosomal mosaicism, or performing linkage analysis.

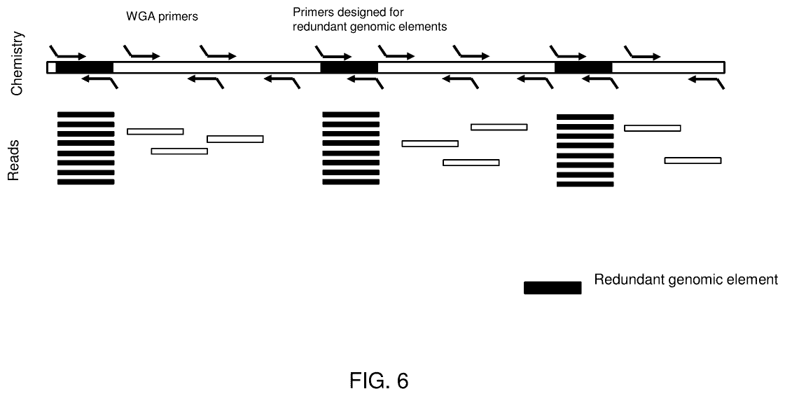

FIG. 6 provides an embodiment of a method for the detection of various mutations, such as SNPs and CNVs, by WGA and targeted amplification of redundant genomic elements. In FIG. 6, quasi random WGA primers provide a shallow and even coverage of the genome and target-specific primers for redundant genomic elements provide robust coverage of SNP-containing regions.

In some cases, the length of WGA/WTA primers and/or target-specific primers can be at least about 5 base pairs (bp), 6 bp, 7 bp, 8 bp, 9 bp, 10 bp, 11 bp, 12 bp, 13 bp, 14 bp, 15 bp, 16 bp, 17 bp, 18 bp, 19 bp, 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, 40 bp, 50 bp, 60 bp, 70 bp, 80 bp, 90 bp, 100 bp, or more.

In some cases, the melting temperature of WGA/WTA primers and/or target-specific primers can be at least about 10° C., 15° C., 20° C., 25° C., 30° C., 35° C., 40° C., 45° C., 50° C., 60° C., 65° C., 70° C., or more. In some cases, WGA/WTA primers can have the same melting temperature as the target-specific primers. In other cases, WGA/WTA primers can have a different melting temperature from the target-specific primers.

In some cases, the GC content of WGA/WTA primers and/or target-specific primers can be at least about 5%, 10%, 15%, 20%, 25%, 30%, 35%, 40%, 45%, 50%, 55%, 60%, or more than 60%. In some cases, WGA/WTA primers can have the same GC content as the target-specific primers. In other cases, WGA/WTA primers can have a different GC content from the target-specific primers.

In some cases, the concentration of WGA/WTA primers and/or target-specific primers can be 1 nanomolar (nM), 10 nM, 20 nM, 30 nM, 40 nM, 50 nM, 60 nM, 70 nM, 80 nM, 90 nM, 100 nM, or more. In some cases, the concentration of WGA/WTA primers and/or target-specific primers can be at least 5 micromolar (μM), 10 μM, 15 μM, 20 μM, 25 μM, 30 μM, 40 μM, 50 μM, 100 μM, 200 μM, 300 μM, 400 μM, 500 μM, 600 μM, 700 μM, 800 μM, 900 μM, or more. In some cases, WGA/WTA primers can have the same primer concentration as the target-specific primers. In other cases, WGA/WTA primers can have a different primer concentration from the target-specific primers.

In some cases, the size of amplicons generated by WGA/WTA primers and/or target-specific primers can be at least about 50 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, or more. In some cases, WGA/WTA primers can generate substantially similar size of amplicons as the target-specific primers. In other cases, WGA/WTA primers can generate substantially different size of amplicons from the target-specific primers. In some cases, WGA/WTA primers can generate substantially similar sizes of amplicons during WGA or WTA. In some cases, WGA/WTA primers can generate substantially different sizes of amplicons during WGA/WTA. In some cases, target-specific primers can generate substantially similar sizes of amplicons during the target-specific amplification of one or more target sequences. In some cases, target-specific primers can generate substantially different sizes of amplicons during the target-specific amplification of one or more target sequences. In some cases, WGA/WTA primers and target-specific primers amplify the same or substantially same region of a genome. For instance, the target-specific primers can be nested within the WGA/WTA primers or vice versa. In some instances, the WGA/WTA primers and the target-specific primers can generate same or substantially same amplicons. For example, the WGA/WTA primers and the target-specific primers may share the same or substantially same binding sites on a nucleic acid molecule.

In some cases, WGA/WTA primers and/or target-specific primers can have different nucleotide sequences. For example, all or substantially all the WGA/WTA primers in a population can have different nucleotide sequences. Similarly, all or substantially all the target-specific primers in a population can have different nucleotide sequences, especially when more than one sequences are targeted, such as in a multiplex reaction.

In some cases, WGA/WTA primers and/or target-specific primers can comprise additional sequences, such as adapter sequences or barcodes such as unique molecular barcodes as described in Winzeler et al. (1999) Science 285:901; Brenner (2000) Genome Biol. 1:1 Kumar et al. (2001) Nature Rev. 2:302; Giaever et al. (2004) Proc. Natl. Acad. Sci. USA 101:793; Eason et al. (2004) Proc. Natl. Acad. Sci. USA 101:11046; and Brenner (2004) Genome Biol. 5:240, each of which also is hereby incorporated by reference in its entirety. For example, WGA/WTA primers can comprise a substantially complete or portion of an Illumina adapter sequence, such as sequences for flow cell attachment sites (e.g., P5, P7), sequences for sequencing primer binding sites (e.g., Read Primer 1, Read Primer 2), index sequences, etc. In some cases, WGA/WTA primers and/or target-specific primers do not comprise any additional sequences. In some other cases, WGA/WTA primers can include additional sequences while target-specific primers do not include any additional sequences. Target-specific primers may include additional sequences, based on the step at which targeted amplification is carried out as well as the number of targeted amplifications performed. For example, if the targeted amplification is carried out in combination with and/or followed by WGA, then the target-specific primers used in the targeted amplification carried out subsequent to WGA may include complete or partial adapter sequences. On the other hand, if the target-specific primers are included in combination with WGA primers in the pre-amplification step, and not in any subsequent steps, then the target-specific primers may include adapter sequences.

In some cases, WGA/WTA primers and/or target-specific primers can have one or more modified nucleotides, such as a locked nucleic acid (LNA), protein nucleic acid (PNA), methylated nucleic acid and the like. In some cases, the modifications may include a nucleic acid with one or more phosphorothioate bond(s), fluorophore(s), biotin, amino-modifiers, thiol modifiers, alkyne modifiers, azide modifiers, spacers, etc. Modified nucleotides may help in cross-linking, duplex stabilization, or nuclease resistance. For example, modified nucleotides may help protect the nucleic acid molecule from the activity of exonucleases or polymerase having an exonuclease activity. In some cases, WGA/WTA primers and/or target-specific primers can have modified nucleotide(s) on one or both ends (e.g., 5′ end, 3′ end) of the oligonucleotide. In some cases, WGA/WTA primers and/or target-specific primers can have modified nucleotide(s) on one end (e.g., 5′ or 3′ end) of the oligonucleotide.

In some cases, WGA/WTA primers and/or target-specific primers can be designed to be substantially non-self-complementary and substantially non-complementary to other primers in the population. For example, WGA/WTA primers can be designed to comprise non-complementary bases, such as guanine (G) and thymine (T) or cytosine (C) and adenine (A), in order to limit interaction of bases in the population, to prevent excessive primer-dimer formation, to reduce complete or sporadic locus dropout, to reduce generation of very short amplification products, and/or to reduce inability to amplify single stranded, short, or fragmented DNA and RNA molecules. In some cases, WGA/WTA primers and/or target-specific primers can have one or more degenerate nucleotide(s) wherein the identify can be selected from a variety of choices of nucleotides, instead of a defined sequence. Degenerate nucleotides may be evenly spaced throughout the WGA/WTA and/or target-specific primers. Degenerate nucleotides can be evenly spaced by including them at specific positions, such as every other base, every second base or every 3^{rd} base, or any other permutation that the experimenter finds useful for their specific application. In other cases, degenerate nucleotides may be restricted to a degenerate or variable region in the primer. An example of a degenerate or variable region may include one or more “N” residues, where N=any base. Such degenerate or variable region can be at a 5′ end and/or 3′ end of the primer sequence. In some cases, the 5′ end may include one or more nucleotides besides non-self-complementary and non-complementary bases. In some cases, the variable or degenerate region of a WGA primer may include adapter sequences, such as Illumina adapter sequences, P5 or P7, for example. In some cases, additional sequences may be included between the constant and the variable or degenerate regions or either end of a WGA/WTA primer.

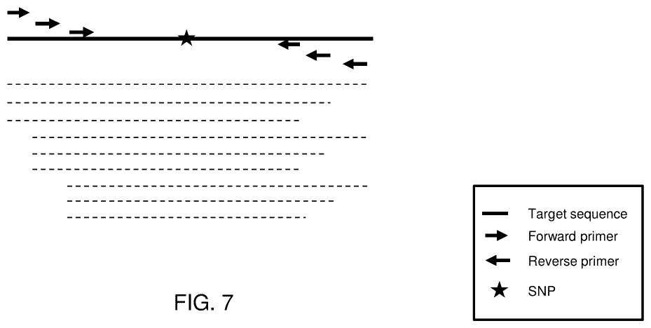

In some cases, WGA/WTA primers and/or target-specific primers can be complementary to adjacent or overlapping positions on the nucleic acid molecule. For example, as shown in FIG. 7, target-specific primers, both forward and reverse, can be designed to be next to each other on the nucleic acid molecule. Such target-specific primers can generate multiple amplicons resulting from various combinations between forward and reverse primers. As shown in FIG. 7, three forward primers and three reverse primers can generate nine distinct amplicons. Such an approach can result in greater amplification of target sequences with mutations, SNPs, for example, which can help better cover the region of interest than the regions not of much interest.

In some cases, WGA/WTA primers and target-specific primers can respectively amplify the whole genome or transcriptome and the target sequence(s) simultaneously, substantially at the same time, or after one another (e.g., WTA/WGA followed by targeted amplification or vice versa) during a pre-amplification step.

In some cases, WGA/WTA and targeted amplification can occur in the same tube, well, cavity, chamber, drop, droplet, solution, reaction, etc. In some cases, the reagents for WGA/WTA and targeted amplification can be mixed together and dispensed into a reaction volume. In some other cases, the reagents for WGA/WTA can be dispensed first into a reaction volume followed by dispensing of the reagents for targeted amplification, or vice versa. In other words, the reagents for targeted amplification can be stacked over the reagents for WGA/WTA. In some cases, targeted amplification and WGA/WTA amplification are carried out simultaneously or substantially simultaneously in the same reaction mixture. In some cases, targeted amplification and WGA/WTA amplification take place sequentially within the same reaction mixture. For example, target-specific primers may amplify their target sequence before WGA/WTA primers amplify their target sequence, or vice versa. In another example, target-specific primers and WGA/WTA primers can amplify their targets substantially at the same time or simultaneously.

In some cases, target-specific primers can be substantially complementary to the target sequence(s). For example, the target-specific primers can be at least about 50%, 65%, 70%, 75%, 80%, 85%, 90%, 95%, 99%, or 100% complementary to the target sequence(s). In some cases, target-specific primers can amplify the target sequence(s) likely comprising mutation(s), such as SNPs. In some cases, target-specific primers can amplify the target sequence(s) comprising more than one mutation, such as two different SNPs. In some cases, target-specific primers can amplify the target sequence(s) comprising more than one different kind of mutation, such as a SNP and an SNV.

In some embodiments, WGA/WTA in combination with targeted amplification can result in a mixture of amplicons comprising WGA/WTA amplicons and targeted amplicons. In some cases, the mixture of amplicons may comprise equal or substantially equal portions of the WGA/WTA amplicons and the targeted amplicons. In some cases, the mixture of amplicons may comprise a larger or substantially larger portion of the WGA/WTA amplicons than the targeted amplicons. For example, the WGA/WTA amplicons can comprise 90% or more while the targeted amplicons can comprise 10% or less of the mixture of amplicons.

In some embodiments, the mixture of amplicons can be directly sequenced on a sequencer. In some cases, the mixture of amplicons can be subjected to a clean-up procedure, a targeted amplification, indexing PCR, and/or any additional amplification procedures prior to sequencing. For example, the mixture of amplicons can be cleaned to remove primers and other reagents (e.g., amplification reagents, lysis reagents, etc.) followed by a nested PCR for amplifying the targeted amplicons prior to sequencing both the WGA amplicons and the targeted amplicons on a sequencer.

### Clean-Up Step

A clean-up step can be performed after cell lysis, or one or more amplification steps. The clean-up step can be useful in removing polymerases, lysis reagents, amplification reagents, primers, unincorporated dNTPs, etc. that can potentially interfere and/or inhibit downstream processes, such as targeted amplification, indexing PCR, a sequencing assay, etc., in an optional clean-up step. The clean-up step can be performed by using any one of the procedures known in the art. For example, the mixture of amplicons generated by WGA in combination with targeted amplification can be cleaned to remove unincorporated dNTPs, amplification reagents, etc. by column-based, gel-based, enzyme-based, and/or bead-based purification techniques.

### Targeted Amplification

Targeted amplification can be carried out in combination with and followed by WGA/WTA in the pre-amplification step. In other cases, the pre-amplification step may include WGA/WTA only and targeted amplification may follow the preamplification step. In some other cases, pre-amplification step may not be carried out and nucleic acid molecules are subjected to targeted amplification to amplify target sequence(s) using target-specific primers.

Targeted amplification carried out in combination with WGA/WTA in the pre-amplification step may generate a mixture of amplicons. This mixture of amplicons can further be amplified using primers nested within the target-specific primers used in the pre-amplification step in a nested PCR. The nested PCR can result in sufficient representation of target sequence(s) for sequencing in a sequencing assay. For example, target sequences that occur in low-frequency can be amplified first in the pre-amplification step using target-specific primers and then in an additional targeted amplification in a nested PCR using nested primers. This would ensure sufficient representation of the target sequences, as indicated by sufficient coverage, determined by the number of unique reads in a sequencing assay. Nested primers may share one or more features with the WGA/WTA primers or target-specific primers. For example, the nested primers may have substantially similar GC content compared to the WGA/WTA primers or target-specific primers. The nested primers may also include adapter sequences (e.g., P5 or P7) as in the WGA/WTA primers, so that the nested amplicons generated can further be amplified by indexing primers to enable sequencing on a sequencing platform, e.g. Illumina. Adapter sequences present in the WGA/WTA or target-specific primers (e.g. nested primers) may include a partial Illumina sequence (e.g. GCTCTTCCGATCT) (SEQ ID NO: 21) or a complete sequence (e.g. AATGATACGGCGACCACCGAGATCTACACXXXXXXXXACACTCTTTCCCTACACGA CGCTCTTCCGATCT) (SEQ ID NO:22), where X=A, C, G or C as part of a barcode index (e.g., a sample index), depending on whether the user wishes to add sequencing indexes indirectly via an indexing PCR step or add the same directly during the additional targeted amplification step. Adapters need not be specific to Illumina sequencing platforms only; the user may modify the adapter sequence to match any appropriate sequence for the sequencing platform of their choice.

In some cases, the length of nested primers used in targeted amplification, either the entire length or the target-specific regions, can be at least about 5 base pairs (bp), 6 bp, 7 bp, 8 bp, 9 bp, 10 bp, 11 bp, 12 bp, 13 bp, 14 bp, 15 bp, 16 bp, 17 bp, 18 bp, 19 bp, 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, 40 bp, 50 bp, 60 bp, 70 bp, 80 bp, 90 bp, 100 bp or more.

In some cases, the melting temperature of nested primers with or without the adapter sequence(s) can be at least about 40° C., 45° C., 50° C., 60° C., 65° C., 70° C., or more. In some cases, nested primers can have the same melting temperature as the target-specific primers. In other cases, nested primers can have a different melting temperature from the target-specific primers.

In some cases, the GC content of nested primers can be at least about 5%, 10%, 15%, 20%, 25%, 30%, 35%, 40%, 45%, 50%, 55%, 60%, or more than 60%. In some cases, nested primers can have the same GC content as the target-specific primers and/or WGA/WTA primers. In other cases, nested primers can have a different GC content from the target-specific primers and/or WGA/WTA primers.

In some cases, the concentration of nested primers in a nested PCR can be at least 1 nanomolar (nM), 10 nM, 20 nM, 30 nM, 40 nM, 50 nM, 60 nM, 70 nM, 80 nM, 90 nM, 100 nM, 5 micromolar (μM), 10 μM, 15 μM, 20 μM, 25 μM, 30 μM, 40 μM, 50 μM, 100 μM, 200 μM, 300 μM, 400 μM, 500 μM, 600 μM, 700 μM, 800 μM, 900 μM, or more.

In some cases, the amplicons generated by nested primers in a nested PCR can be at least about 50 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, or more.

In some cases, nested primers can have one or more modified nucleotides, such as a locked nucleic acid (LNA), protein nucleic acid (PNA), methylated nucleic acid and the like. In some cases, the modifications may include a nucleic acid with one or more phosphorothioate bond(s), fluorophore(s), biotin, amino-modifiers, thiol modifiers, alkyne modifiers, azide modifiers, spacers, Modified nucleotides may help protect the nucleic acid molecule from the activity of exonucleases or polymerase having an exonuclease activity. In some cases, nested primers can have modified nucleotide(s) on one or both ends (e.g., 5′ end, 3′ end) of the oligonucleotide. In some cases, nested primers can have modified nucleotide(s) on one end (e.g., 5′ or 3′ end) of the oligonucleotide.

### Indexing PCR

Either after a pre-amplification reaction or after targeted amplification (e.g., nested PCR), a mixture of amplicons or targeted amplicons can be subjected to an indexing PCR assay to add additional nucleic acid sequence(s), such as Index 1, Index 2, P5, P7, etc., required for performing a sequencing assay on a sequencer. For example, indexing primers comprising Illumina adapter sequences required for compatibility and library clustering on different Illumina sequencers, such as the MiSeq, the NextSeq, the MiniSeq, the HiSeq, the iSeq, the NovaSeq, can be added to the amplicons to generate nucleic acid libraries for further sequencing. Indexing primers comprising barcodes can be used to demultiplex the libraries after pooling in a single run or lane.

### Analysis

After sequencing, the data can be analyzed using custom pipelines to detect variants, such as aneuploidies, copy number variations, etc. In some cases, a pipeline can include functions, such as trimming extra bases (adapter sequences, for example), aligning to a reference sequence (e.g., hg19), sorting and marking duplicate reads, and/or calling variants. In some cases, a pipeline can be customized to accommodate different indexing sequences. In some cases, a shallow and even coverage, as indicated by the number of unique reads, of the genome may be sufficient (e.g., ˜0.025×). In some cases, a robust and deep coverage (e.g., >30×) may be necessary to detect variants, such as SNPs or small indels, etc. In some cases, such as by using a pre-amplification step described herein, a shallow coverage can be used for detecting SNPs or small indels. Sequencing reads may need to be allocated based on the application, such as detection of CNV, SNP, or both.

### Kits

Aspects of the present disclosure also include kits. The kits may include, e.g., a population of primers for WGA/WTA, at least one target-specific primer for targeted amplification, etc. The kits may include a set of instructions for using the kit to detect CNV, genotype SNP, SNV, genotype micro-satellite, detect insertion and/or deletion, determine zygosity, detect gene fusions, detect translocation(s) or detect any other mutation(s). In some cases, a kit may include one or more reagents selected from the group consisting of proteases as thermolysin, alkaline lysis (NaOH), sodium dodecyl sulphate (SDS), triton X-100, digitonin, guanidine, 3-[(3-cholamidopropyl)dimethylammonio]-1-propane-sulphonate, laser pulse, electrical pulse, sonication, Glycerol, 1,2 propanediol, Betaine monohydrate, Tween-20, Formamide, Tetramethyl ammonium chloride (AC), 7-deaza-2′-deoxyguanosine, dimethyl sulfoxide (DMSO), Triton X-100, NP-40, Magnesium, Bovine serum albumin (BSA), ethylene glycol, Dithiothreitol (DTT), KAPA HiFi and KAPA HiFi Uracil+, VeraSeq Ultra DNA Polymerase, VeraSeq 2.0 High Fidelity DNA Polymerase, Takara PrimeSTAR DNA Polymerase, Agilent Pfu Turbo CX Polymerase, Phusion U DNA Polymerase, Deep VentR DNA Polymerase, LongAmp Tag DNA Polymerase, Phusion High-Fidelity DNA Polymerase, Phusion Hot Start High-Fidelity DNA Polymerase, Kapa High-Fidelity DNA Polymerase, Q5 High-Fidelity DNA Polymerase, Platinum Pfx High-Fidelity Polymerase, Pfu High-Fidelity DNA Polymerase, Pfu Ultra High-Fidelity DNA Polymerase, KOD High-Fidelity DNA Polymerase, iProof High-Fidelity Polymerase, High-Fidelity 2 DNA Polymerase, Velocity High-Fidelity DNA Polymerase, ProofStart High-Fidelity DNA Polymerase, Tigo High-Fidelity DNA Polymerase, Accuzyme High-Fidelity DNA Polymerase, VentR DNA Polymerase, DyNAzyme II Hot Start DNA Polymerase, Phire Hot Start DNA Polymerase, Phusion Hot Start High-Fidelity DNA polymerase, Crimson LongAmp Tag DNA Polymerase, DyNAzyme EXT DNA Polymerase, LongAmp Tag DNA Polymerase, Phusion High-Fidelity DNA Polymerase, Tag DNA Polymerase with Standard Taq (Mg-free) Buffer, Tag DNA Polymerase with Standard Tag Buffer, Tag DNA Polymerase with ThermoPol II (Mg-free) Buffer, Tag DNA Polymerase with ThermoPol Buffer, Crimson Taq DNA Polymerase, Crimson Taq DNA Polymerase with (Mg-free) Buffer, Phire Hot Start DNA Polymerase, VentR (exo-) DNA Polymerase, Hemo KlenTaq, Deep VentR (exo-) DNA Polymerase, Deep VentR DNA Polymerase, DyNAzyme EXT DNA Polymerase, Hemo KlenTaq, LongAmp Tag DNA Polymerase, Prot Script AMV First Strand cDNA Synthesis Kit, Prot Script M-MuLV First Strand cDNA Synthesis Kit, Bst DNA Polymerase, Full Length, Bst DNA Polymerase, Large Fragment, 9 Nm DNA Polymerase, DyNAzyme II Hot Start DNA Polymerase, Hemo KlenTaq, Sulfolobus DNA Polymerase IV, Therminator y DNA Polymerase, Therminator DNA Polymerase, Therminator II DNA Polymerase, Therminator III DNA Polymerase, Bsu DNA Polymerase, Large Fragment, DNA Polymerase I (E. coli), DNA Polymerase I, Large (Klenow) Fragment, Klenow Fragment (3′“>5′ exo”), phi29 DNA Polymerase, T4 DNA Polymerase, T7 DNA Polymerase (unmodified), Terminal Transferase, Reverse Transcriptases and RNA Polymerases, E. coli Poly(A) Polymerase, AMV Reverse Transcriptase, M-MuLV Reverse Transcriptase, phi6 RNA Polymerase (RdRP), Poly(U) Polymerase, 5P6 RNA Polymerase, and T7 RNA Polymerase, magnesium salts, nucleotide triphosphate (dNTP) and their derivatives, sodium chloride, potassium chloride, negatively charged carboxyl groups coated magnetic (Polystyrene) beads like AMPure—Beckman Coulter, NucleoMag—MACHEREY-NAGEL, MagJet—ThermoFisher, Mag-Bind—Omega Biotek, ProNex beads—Promega, Kapa Pure Beads—Kapa Biosystems, silica columns like QIAquick PCR Purification Kit and MinElute PCR Purification Kit-Qiagen, PureLink—Thermo Fisher Scientific, GenElute PCR Clean-Up Kit—Sigma, NucleoSpin® Gel and PCR Clean-up—MACHEREY-NAGEL, agarose or acrylamide gels, ethanol or isopropanol precipitation, phenol chloroform extraction, Tris buffer, tween-20, SDS, nucleotide triphosphate (dNTP), Dimethyl sulfoxide, Dimethyl formamide, Tris-HCl pH8.4, ammonium Sulfate, ammonium nitrate, potassium nitrate, TMA-SO4 (Tetramethylammonium sulfate), TMA-Cl (Tetramethylammonium chloride), glycerol, reagents required for sequencing (e.g., MiSeq reagents, NextSeq reagents), Primer oligonucleotides with or without modifications (e.g., LNA, with phosphorothiolated bases), AMPureXP beads, Silica-membrane column, Ethanol, Phenol-chloroform extraction, PEG extraction, or agarose gel.

### Utility

The subject methods find use in the detection of various mutations, such as SNPs, SNVs, CNVs, aneuploidies, translocations, gene fusions, etc. associated genetic disorders. In certain embodiments, the subject methods find use in detecting chromosomal abnormalities and aneuploidies such as, e.g., uniparental disomy, detecting somatic variants in uterine lavage fluid, endometrial fluid to understand the cause of implantation failure or understand the cause of miscarriage, clinical samples, etc. In certain embodiments, the subject methods find use in genomic mapping and genome wide association analyses, e.g., performing SNP-based CNV calling, determining the accuracy of CNV analysis by using SNPs, detecting chromosomal mosaicism, and performing linkage analysis. The subject methods find use in carrier screening for screening individuals suspected of carrying the underlying mutations or known to carry those mutations. The methods find use in screening of embryos (e.g., using a cell or cells of embryos, using culture media in which embryos were cultures, etc.) prior to implantation for detecting mutations associated with genetic disorders. The methods find use in screening fetal DNA or cell-free DNA in maternal samples (e.g., blood, cervix). The methods also find use in determining contamination, such as maternal or paternal DNA or RNA contamination, in embryo biopsies or culture media, such as spent media in which embryos, cells, tissues, or organoids were grown. The subject methods find use in determination of heterozygosity or clonality in a sample. For example, the methods can be used to screen samples such as, tumor biopsies, blood sample, circulating tumor cells, cell-free DNA, or exosomes, for genetic changes such as CNVs and SNP. Such screening may help identify heterogeneity/clonality within tumor cell populations. This may help clinicians to determine treatment options. In some cases, the subject methods find use in human identification applications, forensic applications, DNA fingerprinting, DNA profiling, DNA typing (e.g., during transplantation or engraftment monitoring) or sex determination. In some cases, the subject methods find use in bio-ancestry or genealogical applications, kinship analyses, parentage testing, phylogenetic analyses, or evolutionary studies. In some cases, the subject methods find use in pharmacogenetics and determining the variability in response to pharmacotherapies.

Examples of genetic disorders include, but are not limited to, achondroplasia, adrenoleukodystrophy, alpha thalassaemia, alpha-1-antitrypsin deficiency, Alport syndrome, amyotrophic lateral sclerosis, beta thalassemia, Charcot-Marie-Tooth, congenital disorder of glycosylation type 1a, Crouzon syndrome, cystic fibrosis, Duchenne and Becker muscular dystrophy, dystonia 1, Torsion, Emery-Dreifuss muscular dystrophy, facioscapulohumeral dystrophy, familial adenomatous polyposis, familial amyloidotic polyneuropathy, familial dysautonomia, fanconi anaemia, Fragile X, glutaric aciduria type 1, haemophilia A and B, hemophagocytic lymphohistiocytosis, Holt-Oram syndrome, Huntington's disease, hyperinsulinemic hypoglycemia, hypokalaemic periodic paralysis, Incontinentia pigmenti, Lynch syndrome, Marfan syndrome, Menkes disease, metachromatic leukodystrophy, mucopolysaccharidosis type II (Hunter syndrome), multiple endocrine neoplasia (MEN2), multiple exostosis, myotonic dystrophy, neurofibromatosis type I and II, non-syndromic Sensorineural Deafness, Norrie syndrome, Osteogenesis imperfecta (brittle bone disease), polycystic kidney, autosomal dominant, polycystic kidney, autosomal recessive, Pompe's syndrome, sickle cell anaemia, Smith-Lemli-Opitz syndrome, spastic paraplegia 4, spinal and bulbar muscular atrophy, spinal muscular atrophy, spinocerebellar ataxia 1, 2 and 3, Spondylometaphyseal dysplasia (Schmidt), Tay-Sachs disease, Treacher Collins, tuberous sclerosis, Von Hippel-Lindau syndrome, X-linked dystonia parkinsonism (XDP), X-linked agammaglobulinemia, leukemia, hereditary elliptocytosis and pyropoikilocytosis, autosomal recessive hypercholesterolemia, Fukuyama-type muscular dystrophy. The following example(s) is/are offered by way of illustration and not by way of limitation.

### EXAMPLES

### Example 1: Detection of Copy Number Variation (CNV) and Single Nucleotide Polymorphisms (SNPs)

CNV and SNPs were detected in the same sample using the present disclosure. Briefly, CNV and SNPs were detected in the samples with limited number of cells (for example, single cell or five cells) or genomic DNA (e.g., 30 pg of genomic DNA) using a pre-amplification procedure with WGA/WTA primers in combination with target-specific primers followed by targeted amplification using a nested PCR assay with nested primers and indexing PCR to add sequences required for carrying out a sequencing assay on a sequencer. A next-generation sequencing (NGS) assay was performed to generate sequence reads which were analyzed by custom bioinformatics pipelines for the detection of CNV and SNPs. The method allowed the detection of different mutations at a low sequencing depth of approximately 1 million reads.

The assay was performed using the SMARTer® PicoPLEX® Gold Single Cell DNA-Seq kit (Takara Bio USA, R300669) with some modifications. The kit includes the following steps: cells lysis, whole genome amplification (WGA), DNA purification and addition of Illumina adapters for sequencing compatibility. The kit was modified to amplify certain regions of the CFTR gene using target-specific primers along with WGA. As such, the target-specific primers were added at the pre-amplification step and nested primers were added after the pre-amplification step.

The target-specific primers were designed to amplify specific regions of the genome encompassing variants, such as SNPs or indels of interest and were designed to have a greater specificity to the target sequence than rest of the genome. While designing the target-specific primers, chromosomic locations of other high frequency SNPs that could potentially affect the primer specificity to the target sequence were considered. The target-specific primers generating amplicons of about 600 base pairs (bp) were selected. Multiple target-specific primers were designed and mixed together to target multiple sequences in order to increase likelihood of covering the desired target sequence. The target-specific primers were designed using tools like ThermoBLAST (dnasoftware). A total of 90 target-specific primers were designed to target 15 regions in the CFTR gene, such that 3 primer pairs amplify one target region. The primers were purchased from Integrated DNA Technology (Coralville, Iowa, USA).

First, the effect of number of target-specific primers, such as using 90 target-specific primers, 30 target-specific primers, or no target-specific primers in combination with WGA primers, on the coverage of the CFTR gene was determined. 90 target-specific primers included three primer pairs per target region while 30 target-specific primers included one primer pair per target region. The pre-amplification reaction with the number of target-specific primers with the WGA primers was performed using 30 picograms (pg) of gDNA purchased from the Coriell Institute (Camden, New Jersey, USA). The target-specific primers were included in the PreAmp Buffer and PreAmp Enzyme contained in the SMARTer® PicoPLEX® Gold Single Cell DNA-Seq kit at a concentration of about 20 nM of each target-specific primer.

The pre-amplification reaction was carried out using the below cycling conditions:
Hot start: 95° C. for 3 min—1 cycle
Target-specific amplification: 95° C. for 15 sec, 55° C. for 90 sec, 68° C. for 90 sec—0 to 6 cycles
WGA: 95° C. for 15 sec, 15° C. for 50 sec, 25° C. for 40 sec, 35° C. for 30 sec, 65° C. for 40 sec, 75° C. for 40 sec—14 to 18 cycles.

The number of amplification cycles were adjusted to obtain sufficient quantities of amplicons (e.g., 0.5 to 5 nanograms) for further analysis. If desired, the target-specific amplification can be carried out in a separate reaction from the WGA. In this case, the target-specific primers can efficiently amplify the target sequence(s) due to the optimal cycling conditions. The amplified DNA was then cleaned to remove primers, for example, using AMPure XP beads (Beckman Coulter, cat #A63882).

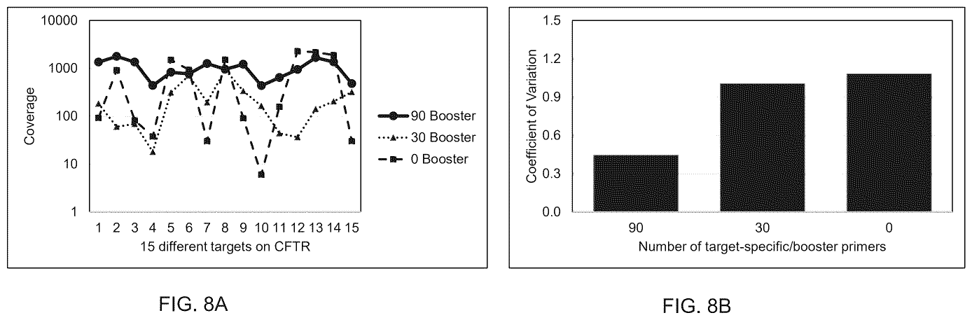

As depicted in FIG. 8A, the coverages for the fifteen different target regions on the CFTR gene were compared among three different primer combinations—0 target-specific primers (0 booster primers), 30 target-specific primers (15 forward and 15 reverse primers; 30 booster primers), and 90 target-specific primers (45 forward and 45 reverse primers; 90 booster primers)—were compared for the coverage and the variations in coverage across the gene. As shown in FIG. 8A, the number of target-specific primers were directly related to the coverage across the CFTR target sequence. X-axis shows the fifteen target regions in the CFTR target sequence. Y-axis shows the number of sequencing reads or coverage, as indicated by the number of unique reads, across the CFTR target gene. For example, a greater coverage across the target sequence was observed when 90 target-specific primers were used compared with 30 or no target-specific primers. Next, as shown in FIG. 8B, 90 target-specific primers reduced the variation in coverage across the CFTR target sequence when compared with 30 or no target-specific primers. In other words, more uniform coverage was observed when 90 target-specific primers were used compared with the coverage when 30 target-specific primers were used. X-axis shows coefficient of variation while Y-axis shows number of primers in each reaction. When 90 target-specific primers were used, the coefficient of variation in the coverage was below 0.5 but when 30 or no target-specific primers were used, the confidence of variation in the coverage was close to 1.

Further, targeted amplification of the CFTR regions was carried out using nested primers in a nested PCR assay. A total of 15 nested primer pairs were designed with each primer comprising 2 functional sections, one at each end, i.e. the 5′ end and 3′ end. The 5′ end section of the primer included Illumina adapter sequences. More specifically, the forward and reverse primers included 13 common bases of the P5 and P7 Illumina adapters. The forward primer included 6 extra bases specific to P5 underlined (read 1): CACGACGCTCTTCCGATCT (SEQ ID NO:23) while the reverse primers included 7 extra bases specific to P7 underlined (read 2); GACGTGTGCTCTTCCGATCT (SEQ ID NO: 24). The 3′end section of the nested primers was designed to amplify segments of the amplicons generated by the target-specific primers in the pre-amplification step. During the selection and design of the nested primers, specificity of the primers was considered. Like the target-specific primers used in the pre-amplification step, the nested target-specific primers were designed using tools like ThermoBLAST (dnasoftware) and the primers with limited affinity to other regions of the genome compared the region of interest were selected. While designing the nested primers, chromosomic locations of other high frequency SNPs that could potentially affect the primer specificity to the target sequence were also considered. The nested primers producing amplicons of about 150 base pairs (bp) were selected. The location of variants, SNPs or indel of interest within the amplicons generated by the nested PCR was considered to make sure that the variants were included in the sequencing reads generated by a sequencer. For example, 2×75 base pair paired end reads were desired, so the nested PCR was performed such that the targeted SNP or mutation was included within the first 75 bases, such as between 15-60, or between 30 to 40 bases from the 3′ end of either of the nested primers used to generate the amplicons. Multiple nested primers were mixed together to multiplex the number of targets amplified. Thirty nested primers at a final concentration of 25 nM were mixed with the Amplification Buffer (reduced Magnesium version) and Amplification Enzyme from the SMARTer® PicoPLEX® Gold Single Cell DNA-Seq kit. The nested PCR assay was carried out using the below cycling conditions:
95° C. for 3 min—1 cycle
95° C. for 30 sec, 56° C. for 2 min, 68° C. for 30 sec—14 cycles

The whole content of the nested PCR step was added to Amplification Buffer and Amplification Enzyme from the SMARTer® PicoPLEX® Gold Single Cell DNA-Seq kit as well as indexing primers SMARTer DNA HT Dual Index Kit—24N (Takara Bio, Cat. No. R400664) or SMARTer DNA Unique Dual Index Kit—24U sets A to D (Takara Bio, Cat. Nos. R400665-R400668) or SMARTer DNA HT Dual Index Kit—96N sets A to D (Takara Bio, Cat. Nos. R400660-R400663). All indexing primers contained essential Illumina adapter sequences required for compatibility and library clustering on different Illumina sequencers, such as the Miseq, the NextSeq, the Miniseq, the HiSeq, the iSeq, or the NovaSeq. The indexing primers also contained barcodes to enable demultiplexing of libraries generated from multiple different samples and sequenced at the same time on the same sequencing run or lane.

The indexing PCR was carried out using the below cycling conditions:
95° C. for 3 min—1 cycle
95° C. for 30 sec, 63° C. for 30 sec, 68° C. for 60 sec—4 cycles
95° C. for 30 sec, 68° C. for 60 sec—6 to 10 cycles

The cycle numbers were adjusted to obtain adequate product yield (e.g., 100 to 500 nanograms) during the indexing PCR. The amplified libraries were cleaned to remove amplification reagents, primers, DNA polymerases and other using AMPure XP beads (Beckman Coulter, cat #A63882) according to the manufacturer's instructions. The libraries were further processed on a MiSeq or NextSeq with 2×75 cycles.

After sequencing, the data was analyzed using custom pipelines. First, the fastq files were down-sampled to 1 million total reads. Adapter sequences and the first 14 bases of the reads were trimmed, and low-quality reads were filtered out using Trimmomatic (Bolger A M, Lohse M and Usadel B., Trimmomatic: a flexible trimmer for Illumina sequence data, Bioinformatics. 2014 Aug. 1; 30(15): 2114-2120). Alignment to the human genome assembly GRCh37 (Church D M et al., Modernizing reference genome assemblies, PLOS Biol. 2011 July; 9(7): e1001091) was then performed with Bowtie2 (Langmead B, Salzberg S. Fast gapped-read alignment with Bowtie 2. Nature Methods. 2012, 9:357-359). Variant calling was performed using Vardict (Lai Z, Markovets A, Ahdesmaki M, Chapman B, Hofmann O, McEwen R, Johnson J, Dougherty B, Barrett J C, and Dry J R. VarDict: a novel and versatile variant caller for next-generation sequencing in cancer research. Nucleic Acids Res. 2016, pii: gkw227).

CNVs can be detected with shallow but uniform coverage while variants, such as SNPs, SNVs, or small indels, may require a deeper coverage. Therefore, to detect CNVs as well as SNPs, SNVs, the number of sequencing reads allocated to the coverage of the whole genome and to the coverage of the target regions in the CFTR gene was optimized. To do so, the coverage of genome and the CFTR gene was compared between two conditions: pre-amplification with WGA and targeted amplification and pre-amplification with WGA without targeted amplification. In both the cases, targeted amplification was carried out after the pre-amplification step. Thirty picogram of genomic DNA was used for the assays.

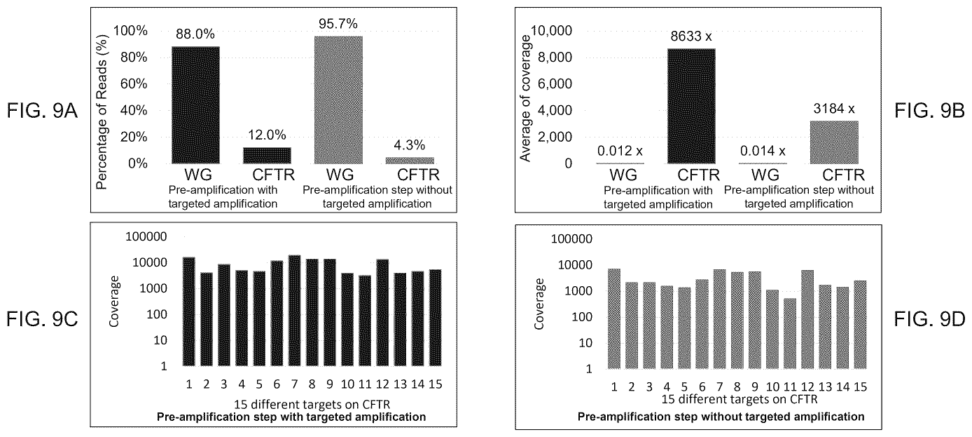

As shown in FIG. 9A, a greater percentage of reads for the CFTR gene was obtained when the pre-amplification step included targeted amplification compared with the pre-amplification step without targeted amplification. X-axis shows the results of the two assays—with and without targeted amplification of the CFTR gene in the pre-amplification step-. Y-axis shows the percentage of reads. A greater percentage of reads (12%) was obtained where the pre-amplification step included targeted amplification compared with the percentage of reads (4.3%) where the pre-amplification step did not include targeted amplification. On the contrary, a greater percentage reads (95.7%) from the WGA was observed when no targeted amplification was included in the pre-amplification step compared with the reads from the assay when the pre-amplification step included targeted amplification (88%). As shown in FIG. 9B, a greater coverage, as indicated by the number of unique reads, of the CFTR gene (8633×) was observed with the assay with targeted amplification in the pre-amplification step when compared with the coverage obtained with the assay without targeted amplification (3184×) in the pre-amplification step. X-axis shows two different assays—with and without targeted amplification of the CFTR gene in the pre-amplification step. Y-axis shows the average coverage. Further, the uniformity of coverage across the fifteen different regions in the CFTR gene was improved in the assay with targeted amplification included in the pre-amplification step (FIG. 9C) when compared with the coverage without targeted amplification included in the pre-amplification step (FIG. 9D). X-axis shows 15 different target regions in the CFTR gene and Y-axis shows coverage or the number of unique reads, at each target region.

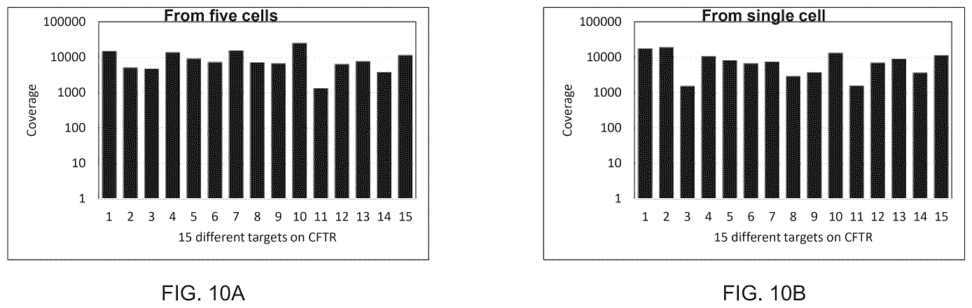

The uniformity of coverage across the fifteen amplicons of the CFTR gene was assessed using single cells (n=4) and five (n=4) sorted cells and using 90 target-specific primers in the pre-amplification step followed by targeted amplification and indexing PCR to generate library molecules for sequencing. More uniform coverage across the fifteen target regions in the CFTR gene was observed in the five-cells samples compared with the single cell samples, as shown in FIG. 10A and FIG. 10B. However, the uniformity of coverage was fully acceptable in both the sample types for further analysis.

Next, sequencing reads were analyzed for detecting variants in the CFTR gene. As shown in Table 1, different variants and their allele frequencies were detected using single cells or five sorted cells for both GM07552 and GM012785. GM07552 cells contain known variants—Phe508DEL, Arg553TER and has alleles 7T/9T in the CFTR gene. GM12785 cells contain ARG347PRO, GLY551ASP, 7T/7T known variants in the CFTR gene. For the experiments performed with five sorted cells using GM07552 or GM12785, all the heterozygous variants were identified correctly at an allele frequency between 0.2 and 0.8. When all bases covered by the panel (2,250 bases) were interrogated, no other variants were reported above an allele frequency of 0.1. The false positive rate was virtually 0%. Similarly, heterozygous variants were identified using single cells.

TABLE 1

| GM07552 | | | | GM12785 | | | |
|---|---|---|---|---|---|---|---|
| | delF508 | Arg553ter | 7T/9T | | Arg347Pro | Arg551Asp | 7T/7T |
| From five cells | | | | From five cells | | | |
| Replicate 01 | 0.52 | 0.58 | 0.43/0.57 | Replicate 01 | 0.46 | 0.52 | 1 |
| Replicate 02 | 0.43 | 0.58 | 0.30/0.70 | Replicate 02 | 0.54 | 0.58 | 1 |
| Replicate 03 | 0.49 | 0.63 | 0.29/0.71 | Replicate 03 | 0.68 | 0.51 | 1 |
| Replicate 04 | 0.44 | 0.63 | 0.30/0.70 | Replicate 04 | 0.64 | 0.58 | 1 |
| From single cell | | | | From single cell | | | |
| Replicate 01 | 0.64 | 0.64 | 0.43/0.57 | Replicate 01 | 0.19 | 0.49 | 1 |
| Replicate 02 | 0.21 | 0.98 | 0.31/0.69 | Replicate 02 | 0.97 | 0.30 | 1 |
| Replicate 03 | 0.53 | 0.27 | 0.45/0.55 | Replicate 03 | 0.97 | 0.59 | 1 |
| Replicate 04 | 0.51 | Low coverage | 0.30/0.70 | Replicate 04 | 0.74 | 0.76 | 1 |

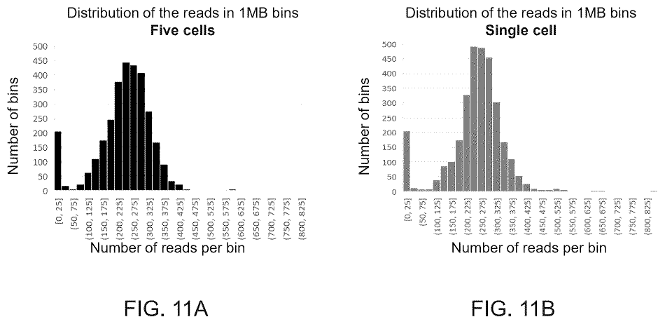

The distribution of sequencing reads in 1 Mb bins was determined using GM12785-five cells or single cell as respectively shown in FIG. 11A and FIG. 11B. As shown FIG. 11A and FIG. 11B, the number of reads per bin shows similar patterns between the five-cell sample and the single-cell sample across various bins, demonstrating sensitivity and reproducibility of the assay.

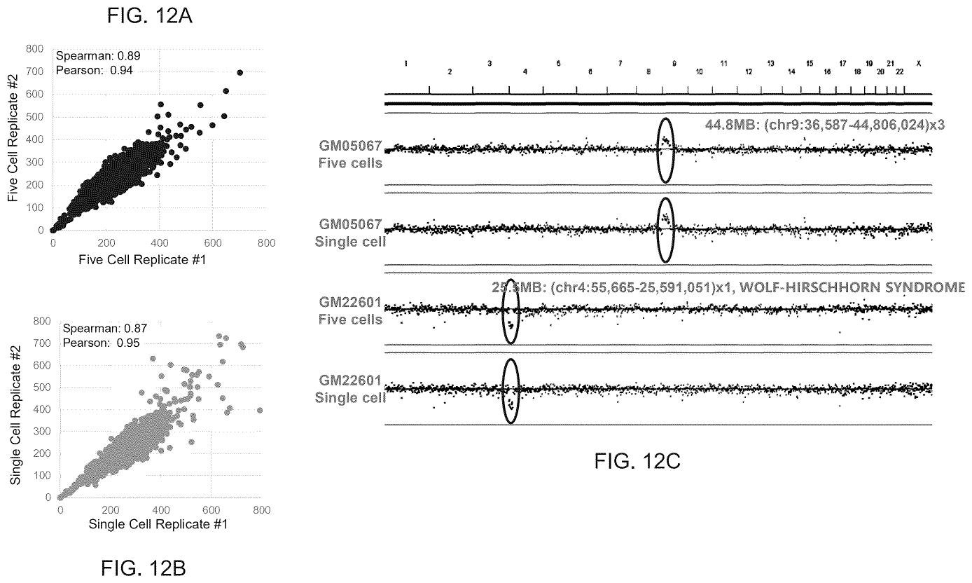

The reproducibility of the read distribution between replicates of five sorted cells (N=4), as shown in FIG. 12A and single cell (N=4), as shown in FIG. 12B, of GM12785 was assessed by calculating the Pearson and Spearman correlations. A strong correlation was observed for both the five-cells and the single cell replicates, demonstrating the robustness of the whole genome amplification even in the presence of the targeted primers. The log 2 ratio for each bin was calculated between two replicates of the five GM12785 sorted cells or single cells and was plotted using IGV (Broad Institute), as shown in FIG. 12C. As depicted, the bins were conserved between the two replicates, and expected copy number variations were observed in Chr. 9 and Chromosome 4, respectively, in GM05067 and GM22601.

In summary, the addition of target-specific primers and nested primers to the SMARTer® PicoPLEX® Gold Single Cell DNA-Seq enabled robust and even coverage of the genome, as well as deep coverage of the fifteen key regions of the CFTR gene from single or five cells in a single tube workflow. The assay performed well when using a total of 1 Million reads. When using five sorted cells, the detections of five different characterized heterozygous mutations was virtually 100%. No false positive were detected in the 2,250 bases panel.

### Example 2: Targeted Amplification for SNP Detection

In this example, we demonstrated the use of target-specific primers for the detection of SNPs, in carrier screening, for example. 15 ng of genomic DNA, NA07552 or NA012785, was respectively extracted from GM07552 or GM12785 cells. GM07552 cells contain the following known variants of CFTR: Phe508DEL, Arg553TER and has alleles 7T/9T. GM12785 contain the following known variants in the CFTR gene: Arg347Pro, Gly551Asp, and has alleles 7T/7T. The extracted genomic DNA, NA07552 or NA012785, was subjected to targeted amplification using 15 pairs target-specific primers to amplify 15 different variants in the CFTR gene. The target-specific primers, at a final concentration of 25 nM, were mixed with the Amplification Buffer (reduced Magnesium version) and Amplification Enzyme from the SMARTer® PicoPLEX® Gold Single Cell DNA-Seq kit. The targeted amplification PCR was carried out as follows:
95° C. for 3 min—1 cycle
95° C. for 30 sec, 56° C. for 2 min, 68° C. for 30 sec—14 cycles

The content of the targeted amplification was added to Amplification Buffer and Amplification Enzyme from the SMARTer® PicoPLEX® Gold Single Cell DNA-Seq kit as well as indexing primers SMARTer DNA HT Dual Index Kit−24N (Takara Bio, Cat. No. R400664) or SMARTer DNA Unique Dual Index Kit−24U sets A to D (Takara Bio, Cat. Nos. R400665-R400668) or SMARTer DNA HT Dual Index Kit−96N sets A to D (Takara Bio, Cat. Nos. R400660-R400663). All indexing primers included the Illumina adapter sequences required for compatibility and library clustering on different Illumina sequencers as the Miseq, the NextSeq, the Miniseq, the HiSeq, the iSeq, the NovaSeq. The indexing primers also contained barcodes that were used to demultiplex the libraries after pooling in a single run.

The indexing PCR was carried out as follows:
95° C. for 3 min—1 cycle
95° C. for 30 sec, 63° C. for 30 sec, 68° C. for 60 sec—4 cycles
95° C. for 30 sec, 68° C. for 60 sec—6 cycles

The amplified libraries were cleaned to remove amplification reagents, primers, DNA polymerases and other using AMPure XP beads (Beckman Coulter, cat #A63882).

The libraries were further processed on MiSeq 2×75 cycles.

After sequencing, the data was analyzed using custom bioinformatics pipelines. First, the fastq files were down-sampled to 1 million total reads. Adapter sequences and the first 14 bases of the reads were trimmed, and low-quality reads were filter out using Trimmomatic (Bolger A M, Lohse M and Usadel B., Trimmomatic: a flexible trimmer for Illumina sequence data, Bioinformatics. 2014 Aug. 1; 30(15): 2114-2120). Alignment to the human genome assembly GRCh37 (Church D M et al., Modernizing reference genome assemblies, PLOS Biol. 2011 July; 9(7): e1001091) was subsequently performed with Bowtie2 (Langmead B, Salzberg S. Fast gapped-read alignment with Bowtie 2. Nature Methods. 2012, 9:357-359). Variant calling was performed using Vardict (Lai Z, Markovets A, Ahdesmaki M, Chapman B, Hofmann O, McEwen R, Johnson J, Dougherty B, Barrett J C, and Dry J R. VarDict: a novel and versatile variant caller for next-generation sequencing in cancer research. Nucleic Acids Res. 2016, pii: gkw227).

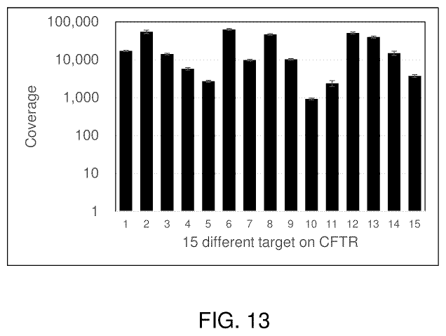

As shown in FIG. 13, a uniform coverage across the fifteen target regions in the CFTR gene was observed with targeted amplification alone using target-specific primers described in the present disclosure. X-axis shows the fifteen target regions or variants in the CFTR gene. Y-axis shows the coverage, as indicated by the number of unique reads, for each of the target regions. Further, as shown in Table 2, using targeted amplification alone, we were able to identify all the five heterozygous variants correctly at an allele frequency between 0.4 and 0.6. When all bases covered by the panel (2,250 bases) were interrogated, no other variants were reported above an allele frequency of 0.05. The false positive rate was virtually 0%.

TABLE 2

| GM07552 | | | | GM12785 | | | |
|---|---|---|---|---|---|---|---|
| From 15 ng gDNA | | | | From 15 ng gDNA | | | |
| | delF508 | 1 Arg553ter | 7T/9T | | Arg347Pro | Arg551Asp | 7T/7T |
| Replicate 01 | 0.45 | 0.51 | 0.43/0.57 | Replicate 01 | 0.49 | 0.48 | 1 |
| Replicate 02 | 0.52 | 0.50 | 0.42/0.58 | Replicate 02 | 0.48 | 0.48 | 1 |
| Replicate 03 | 0.50 | 0.50 | 0.40/0.60 | Replicate 03 | 0.49 | 0.49 | 1 |

Based on this experiment, we conclude that targeted amplification can be used to detect SNPs, especially where WGA is not required or where a large amount of input DNA is available. One such example may include the detection of SNPs in carrier screening for parents.

### Example 3: Detection of CFTR Mutations in Clinical Samples

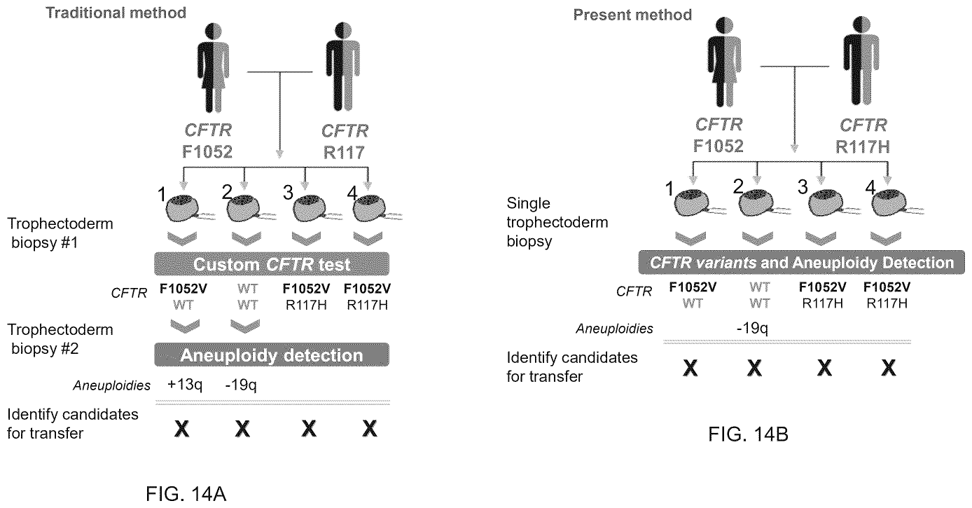

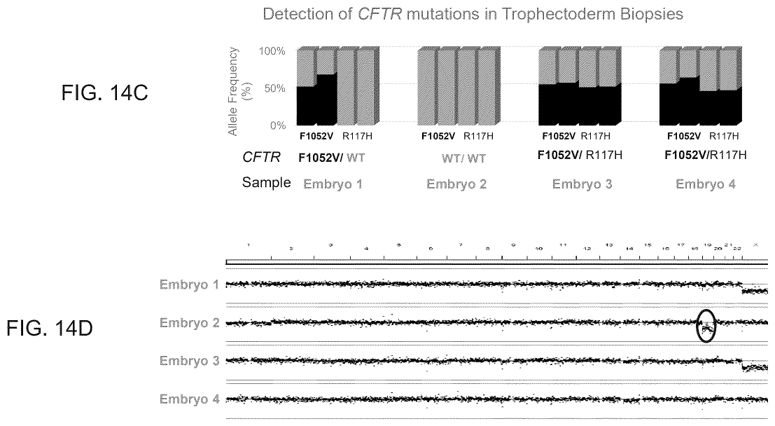

This study was done using trophectoderm biopsy samples that were collected from embryos that had previously been subjected to traditional SNP and CNV analysis using a two-step method whereby a first biopsy was used for SNP determination and a second biopsy was then used to determine copy number. This is outlined schematically in FIG. 14A. The 4 embryos came from a mother determined to be a carrier for the pathogenic CFTR variant SNP, F1052V, and a father determined to be a carrier for the R117H variant. As shown in FIG. 14A, the first biopsy revealed embryos 3 and 4 to be compound heterozygotes, carrying the pathogenic variants from both mother and father. These two embryos were thus not further screened for potential copy number variation (CNV) using a second biopsy. Embryos 1 and 2 were carried forward for a second biopsy and potential CNV aneuploidies were identified in embryos 1 and 2.

In this example, using the methods described in the present disclosure, a third biopsy was taken from the same 4 embryos and used to show how the methods of the current disclosure can identify both SNP and CNV abnormalities from a single biopsy test. This is shown schematically in FIG. 14B. Trophectoderm biopsy samples (n=4), each containing approximately 5 cells, were taken and subjected to the methods of the current disclosure using combination with WGA and targeted amplification. The experiment was repeated twice from the same biopsy samples, and the results are shown in FIG. 14C. As shown in the top panel (FIG. 14C), the zygosity of each of the 4 embryos for the maternal and paternal pathogenic CFTR variants was assessed and found to concur with the traditional method on both occasions, revealing embryos 3 and 4 to be compound heterozygotes, embryo 1 to be a carrier for the maternal CFTR variant and embryo 2 to be wild-type. In the lower panel (FIG. 14D) is also shown the CNV analysis of the 4 embryos from one of the pair of assays that was run. This revealed that embryos 1, 3 and 4 had normal karyotypes whereas embryo 2 showed a partial loss of Chromosome 19q, confirming the result obtained by the traditional 2 step method. Embryo 1 was found to be wild type. It is possible that this reflects mosaicism in the embryo. In conclusion, we show that our combined WGA/targeted sequencing method allows determination of SNP and CNV alterations from a single embryo biopsy; thus, improving utility over traditional two-step methods that assess SNP and CNV separately.

### Example 4: Detection of Variants in SVA Elements

SNPs and CNVs were detected using samples of human genomic DNA and a pre-amplification procedure including a single target-specific primer pair for amplifying a redundant genomic element in combination with primer pairs for whole genome amplification. SVA elements were selected as a candidate redundant genomic element as they are found on all autosomes and sex chromosomes at a density that would allow for SNP-based analysis on all chromosomes (Table 3).

TABLE 3
Number of SVA elements and their average occurrence across the genome.

| Chromosome | Number of SVA Elements | SVA Element Density (bp) |
|---|---|---|
| 1 | 13,944 | 16,529 |
| 2 | 8,639 | 27,844 |
| 3 | 7,222 | 27,430 |
| 4 | 4,687 | 40,485 |
| 5 | 6,148 | 29,484 |
| 6 | 5,854 | 29,053 |
| 7 | 9,182 | 17,313 |
| 8 | 4,614 | 31,376 |
| 9 | 5,861 | 20,780 |
| 10 | 6,207 | 21,470 |
| 11 | 5,972 | 22,527 |
| 12 | 8,100 | 16,437 |
| 13 | 2,443 | 40,108 |
| 14 | 4,348 | 20,830 |
| 15 | 4,630 | 18,281 |
| 16 | 8,012 | 10,210 |
| 17 | 10,004 | 8,289 |
| 18 | 2,243 | 35,706 |
| 19 | 12,884 | 4,536 |
| 20 | 4,295 | 14,888 |
| 21 | 1,320 | 30,370 |
| 22 | 4,336 | 9,031 |
| X | 5,094 | 30,407 |
| Y | 1,058 | 24,967 |
| Whole | 147,097 | 19,971 |
| Genome | | |

Number of SVA elements and their locations were accessed from the Dfam database of repetitive DNA families using the hg38 human genome assembly. SVA element density is based off of the mappable portions of each chromosome using the hg38 human genome assembly.

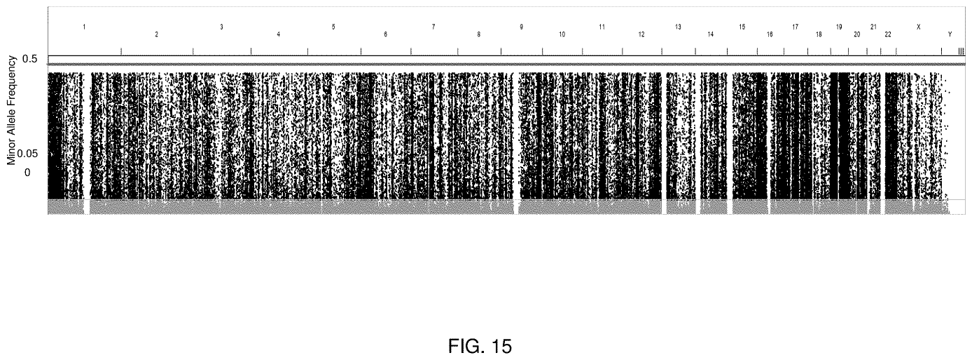

To determine the number of SNPs contained in these SVA elements, the latest release of the human SNP database from the National Center for Biotechnology Information was used as a reference to determine the total number of SNPs and number of informative SNPs (minor allele frequency >=0.05) found within SVA elements (Table 4). There are an estimated 146,856 informative SNPs found within 10 SVA elements occurring on average once every 67,109 bp. Informative SNPs occur within SVA elements across all chromosomes (FIG. 15).

TABLE 4
Number of SNPs within SVA elements and their average
occurrence across the genome.

| | Number of SNPs | | SNP density (SNP/bp) | |
|---|---|---|---|---|
| | | Informative | | Informative |
| Chromosome | All | (AF > 0.05) | All | (AF > 0.05) |
| 1 | 46,111 | 13,177 | 4,998 | 17,491 |
| 2 | 28,315 | 8,110 | 8,495 | 29,661 |
| 3 | 23,688 | 6,934 | 8,363 | 28,569 |
| 4 | 17,909 | 5,457 | 10,595 | 34,772 |
| 5 | 21,266 | 6,134 | 8,524 | 29,551 |
| 6 | 21,908 | 6,959 | 7,763 | 24,440 |
| 7 | 30,237 | 8,960 | 5,257 | 17,742 |
| 8 | 16,611 | 4,732 | 8,715 | 30,593 |
| 9 | 20,015 | 5,870 | 6,085 | 20,748 |
| 10 | 21,085 | 6,351 | 6,320 | 20,983 |
| 11 | 20,110 | 5,844 | 6,690 | 23,021 |
| 12 | 27,235 | 8,259 | 4,888 | 16,120 |
| 13 | 9,113 | 2,915 | 10,752 | 33,613 |
| 14 | 15,321 | 4,483 | 5,911 | 20,203 |
| 15 | 14,988 | 4,293 | 5,647 | 19,716 |
| 16 | 25,330 | 7,178 | 3,230 | 11,397 |
| 17 | 32,408 | 9,506 | 2,559 | 8,723 |
| 18 | 8,381 | 2,507 | 9,556 | 31,946 |
| 19 | 46,147 | 14,685 | 1,266 | 3,980 |
| 20 | 13,240 | 3,961 | 4,830 | 16,143 |
| 21 | 4,219 | 1,399 | 9,502 | 28,655 |
| 22 | 14,010 | 4,436 | 2,795 | 8,828 |
| X | 15,999 | 4,682 | 9,681 | 33,083 |
| Y | 174 | 24 | 151,811 | 1,100,627 |
| Total | 493,820 | 146,856 | | |
| Mean per | 20,576 | 6,119 | 12,676 | 67,109 |
| Chromosome | | | | |

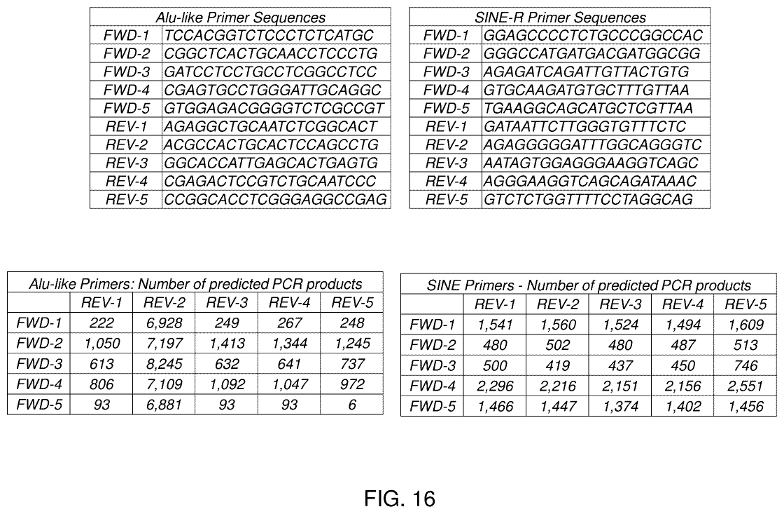

SVA elements contain seven distinct regions (FIG. 5). Target-specific primer pairs were designed to amplify regions of SVA elements such as the Alu-like or SINE-R regions. Fifty candidate target-specific primers were screened for their capacity to amplify the targeted SVA elements. The target-specific primers were designed using tools such as the BiSearch Primer Design and Search Tool (FIG. 16) (Aranyi et al., (2006)). 25 different primer pair combinations of forward and reverse primers, disclosed in FIG. 16, were tested for each region, namely the Alu-like or SINE-R regions. A total of 50 primer pair combinations were tested, and the target-specific primers that 10 successfully amplified their target region and produced an amplicon product near their predicted size were selected for incorporation into the pre-amplification step of the WGA/WTA methods as provided in the present disclosure. Out of the fifty primer pairs, a total of 37 SVA specific primer pairs were selected.

In summary, incorporation of SVA-specific primer pairs into the pre-amplification step of the whole genome amplification process amplifies SNP containing regions of the SVA element at a density and distribution across the human genome to perform SNP-based analyses described in detail in the Methods section of this patent application.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

What is claimed is:

1. A method of detecting single nucleotide polymorphism (SNP) and copy number variation (CNV) in a sample, the method comprising:
a) obtaining a sample comprising nucleic acid molecules;
b) subjecting the nucleic acid molecules to a population of primers for whole genome amplification or whole transcriptome amplification and to at least one target-specific primer for targeted amplification to generate a mixture of amplicons produced by the whole genome amplification or whole transcriptome amplification and the targeted amplification;
c) sequencing the mixture of amplicons using a sequencing assay on a sequencer to generate sequencing reads; and
d) assessing the sequencing reads to determine the SNP and CNV in the sample.

2. The method of claim 1, wherein the nucleic acid molecules are amplified by using a polymerase chain reaction.

3. The method of claim 1, wherein the mixture of amplicons produced in step (b) is subjected to an additional targeted amplification using at least one nested primer pair to further amplify amplicons generated by the targeted amplification.

4. The method of claim 1, the method further comprises using the sequencing reads to genotype single nucleotide variation (SNV), genotype micro-satellite, detect insertion and/or deletion, determine zygosity, determine sex, detect gene fusions, detect translocation(s), detect mutation(s), or detect chromosomal abnormalities.

5. The method of claim 1, wherein the population of primers are non-self-complementary and non-complementary to other primers in the population, and comprise in a 5′ to 3′ orientation a constant region and a variable region, wherein the constant region sequence has a known sequence that is constant among a plurality of primers of the population and the variable region sequence is degenerate among the plurality of primers of the population, and further wherein the sequence of the constant and variable regions consists will not cross-hybridize or self-hybridize under conditions to carry out steps (a)-(c).

6. The method of claim 1, wherein the plurality of nucleic acid molecules is at least 50 base pairs.

7. The method of claim 1, wherein the primers as in (b) comprise at least 10 nucleotides.

8. The method of claim 1, wherein the at least one target-specific primer is specific to one or more target sequences.

9. The method of claim 1, wherein the at least one target-specific primer does not comprise an adapter sequence.

10. The method of claim 1, wherein the at least one target-specific primer comprises at least a portion of an adapter sequence.

11. The method of claim 1, wherein the primers as in (b) comprises at least one modified nucleotide.

12. The method of claim 1, wherein melting temperature of the primers as in (b) is at least 30 degrees Celsius.

13. The method of claim 1, wherein the nucleic acid molecules comprise genomic DNA, or RNA.

14. The method of claim 1, wherein the sample is selected from the group consisting of blood, serum, plasma, cerebrospinal fluid, cheek scrapings, nipple aspirate, biopsy, cervical sample, semen, bodily fluid, microorganisms, mitochondria, chloroplasts, a cell lysate, urine, feces, hair follicle, saliva, sweat, immunoprecipitated or physically isolated chromatin, circulating tumor cells, tumor biopsy samples, exosomes, embryo, cell culture medium, spent medium for culturing cells, tissues, organoids, or embryos, biopsied embryo, trophoblast, amniotic fluid, maternal blood, fetal cell, fetal DNA, cell-free DNA, uterine lavage fluid, endometrial fluid, cumulus cells, granulosa cells, formalin-fixed tissue, paraffin-embedded tissue or blastocoel cavity.

15. The method of claim 9, wherein the at least one target-specific primer comprises a single target-specific primer pair.

16. The method of claim 15, wherein the one or more target sequences comprise a redundant genomic region.

17. The method of claim 16, wherein the redundant genomic region comprises a repetitive element.

18. The method of claim 17, wherein the repetitive element comprises an SVA element.

19. A kit, comprising:
a) a population of primers for whole genome amplification or whole transcriptome amplification;
b) at least one target-specific primer for targeted amplification; and
d) a set of instructions for using the kit to detect copy number variation (CNV), genotype single nucleotide polymorphism (SNP), detect single nucleotide variation (SNV), genotype micro-satellite, detect insertion and/or deletion, determine zygosity, determine sex, detect gene fusions, detect translocations, detect mutation(s), or detect chromosomal abnormalities.
